(19) United States
(12) Patent Application Publication
(10) Pub. No.: US 2025/0188167 A1
(43) Pub. Date: Jun. 12, 2025

(54) ANTIBODIES AND VARIANTS THEREOF AGAINST HUMAN B7-H3

(71) Applicant: Nanjing Probio Biotech Co., Ltd., Nanjing, Jiangsu (CN)

(72) Inventors: Yi Zhou, Nanjing, Jiangsu (CN); Zhongdao Li, Nanjing, Jiangsu (CN); Zhuo Fang, Nanjing, Jiangsu (CN); Wenshuang Jia, Nanjing, Jiangsu (CN); Cuiying Shao, Nanjing, Jiangsu (CN)

(73) Assignee: Nanjing Probio Biotech Co., Ltd., Nanjing, Jiangsu (CN)

(21) Appl. No.: 18/841,170

(22) PCT Filed: Feb. 7, 2023
(86) PCT No.: PCT/CN2023/074767

§ 371 Date: Aug. 23, 2024

(30) Foreign Application Priority Data
Feb. 25, 2022 (WO) PCT/CN2022/078008

(51) Int. Cl. C07K 16/28; A61K 47/68; A61P 35/00

(52) U.S. Cl. CPC C07K 16/2827; A61K 47/68031; A61K 47/68033; A61K 47/6821; A61K 47/6829; A61P 35/00; C07K 2317/24; C07K 2317/31; C07K 2317/515; C07K 2317/52; C07K 2317/565; C07K 2317/732; C07K 2317/92

(57) ABSTRACT

The present application provides an antibody, such as a monoclonal antibody (mAb), or an antigen binding fragment thereof, that specifically recognizes B7-H3. Also provided are pharmaceutical compositions, or methods of making and using the antibody or antigen-binding fragment thereof.

### CROSS-REFERENCE TO RELATED APPLICATIONS

This is the U.S. National Stage of PCT Application No. PCT/CN2023/074767, filed Feb. 7, 2023, which was published in English under PCT Article 21(2), and claims priority to PCT application No. PCT/CN2022/078008, filed on Feb. 25, 2022. The entire contents of these applications are incorporated herein by reference for all purposes.

### INCORPORATION OF SEQUENCE LISTING

The Sequence Listing is submitted as an XML file named “Sequence Listing_112457-01.xml,” created on Aug. 22, 2024 (˜85,214 bytes), which is incorporated herein by reference.

### FIELD OF THE INVENTION

The application relates to antibodies or antigen binding fragments thereof capable of binding specifically to a B7-H3 protein and uses of such agents. The antibodies or antigen binding fragments thereof are useful for the treatment of diseases associated with the activity and/or expression of B7-H3.

### BACKGROUND

B7-H3 is a single-pass type I transmembrane protein discovered in 2001, which belongs to the B7 family [Molecular characterization of human 4Ig-B7-H3, a member of the B7 family with four Ig-like domains. J Immunol 172(4):2352-2359]. It is widely expressed in various cancers, including non-small cell lung cancer (NSCLC), head and neck squamous cell carcinoma (HNSCC), breast cancer, and others, but low or undetectable in normal tissue [B7-H3: an attractive target for antibody-based immunotherapy. Clin Cancer Res]. Meanwhile, B7-H3 has been found in low or negative PD-L1 expressing solid tumors. More than 20 clinical trials on B7-H3 have been carried out, including monoclonal antibodies (Mabs), bispecific antibodies (BsAbs), antibody-drug conjugates (ADCs), chimeric antigen receptor (CAR)-T and radioimmunotherapy (RIT).

MGC018 (NCT03729596) and DS-7300a (NCT04145622), two ADCs against B7-H3, are tested in phase I/II clinical study. While ADCs targeting B7-H3 whether cause life-threatening off-tumor on-target toxicity is unclear, owing to B7-H3's low expressing in normal cell [New B7 family checkpoints in human cancers. Mol Cancer Ther 16(7):1203-1211]. Targeting B7-H3 RIT (iodine-131 labeled omburtamab) has been granted breakthrough therapy, but up to now, its indication is almost limited to brain tumors.

MGA271 (Enoblituzumab) and DS-5573a are two antibodies targeting B7-H3. MGA271 (Enoblituzumab, Macrogenics) is an Fe-optimized humanized IgG1 antibody, and its safety profile and efficacy have been tested in three clinical studies (NCT01391143, NCT02923180, NCT02982941), which have the potency of B7-H3-dependent antibody dependent cellular cytotoxicity (ADCC) activity. DS-5573a (Daiichi Sankyo Inc) is a humanized anti-B7-H3 IgG1 [Development of DS-5573a: a novel afucosylated mAb directed at B7-H3 with potent antitumor activity. Cancer Sci 107(5):674-681]. Clinical trial on DS-5573a is an open-label phase I study (NCT021925679), which was initiated in 2014 with advanced solid tumors patients, and it was terminated on the business decision in 2017. DS-5573a kill tumor cells depend on ADCC and complement dependent cytotoxicity (CDC) activity.

Avelumab (anti-PD-L1), trastuzumab (anti-HER2) and cetuximab (anti-EGFR) are monoclonal antibodies, which kill tumor cells largely depends on ADCC. These approved drugs support the therapeutic potential of ADCC-related drugs in solid tumor treatment. Additional, according to the clinical results of NCT02923180 and NCT02982941, MGA271 (Enoblituzumab) showed the remission of patients with B7-H3-expressing relapsed or refractory solid tumors.

There is a need to develop antibodies targeting B7-H3, which could kill tumor such as NSCLC and HNSCC depend on ADCC and CDC, and have better efficacy than positive control MGA271 (Enoblituzumab) and DS-5573a.

### SUMMARY

In one aspect, the present disclosure provides an isolated antibody or an antigen-binding fragment thereof, comprising:
I) a heavy chain variable domain (VH) comprising:
1) a heavy chain determining region 1 (CDR1) comprising an amino acid sequence selected from the group consisting of SEQ ID NOs: 2, 10, 18, 26, 34, 42, 50, 58, 66, and 74;
2) a heavy chain CDR2 comprising an amino acid sequence selected from the group consisting of SEQ ID NOs: 3, 11, 19, 27, 35, 43, 51, 59, 67, and 75; and
3) a heavy chain CDR3 comprising an amino acid sequence selected from the group consisting of SEQ ID NOs: 4, 12, 20, 28, 36, 44, 52, 60, 68, and 76, respectively; and
II) a light chain variable domain (VL) comprising:
1) a light chain CDR1 comprising an amino acid sequence selected from the group consisting of SEQ ID NOs: 6, 14, 22, 30, 38, 46, 54, 62, 70, and 78;
2) a light chain CDR2 comprising an amino acid sequence selected from the group consisting of SEQ ID NOs: 7, 15, 23, 31, 39, 47, 55, 63, 71, and 79; and
3) a light chain CDR3 comprising an amino acid sequence selected from the group consisting of SEQ ID NOs: 8, 16, 24, 32, 40, 48, 56, 64, 72, and 80, respectively,
wherein the antibody or antigen-binding fragment thereof is capable of specifically binding to a B7-H3, preferably a humanB7-H3.

In some embodiments, in the isolated antibody or an antigen-binding fragment thereof, 1) the VH comprises the heavy chain CDR1, CDR2, and CDR3 sequences having the amino acid sequences of SEQ ID NOs: 2, 3, and 4, respectively, and the VL comprises the light chain CDR1, CDR2, and CDR3 having the amino acid sequences of SEQ ID NOs: 6, 7, and 8, respectively; 2) the VH comprises the heavy chain CDR1, CDR2, and CDR3 sequences having the amino acid sequences of SEQ ID NOs: 10, 11, and 12, respectively, and the VL comprises the light chain CDR1, CDR2, and CDR3 having the amino acid sequences of SEQ ID NOs: 14, 15, and 16, respectively; 3) the VH comprises the heavy chain CDR1, CDR2, and CDR3 sequences having the amino acid sequences of SEQ ID NOs: 18, 19, and 20, respectively, and the VL comprises the light chain CDR1, CDR2, and CDR3 having the amino acid sequences of SEQ ID NOs: 22, 23, and 24, respectively; 4) the VH comprises the heavy chain CDR1, CDR2, and CDR3 sequences having the amino acid sequences of SEQ ID NOs: 26, 27, and 28, respectively, and the VL comprises the light chain CDR1, CDR2, and CDR3 having the amino acid sequences of SEQ ID NOs: 30, 31, and 32, respectively; 5) the VH comprises the heavy chain CDR1, CDR2, and CDR3 sequences having the amino acid sequences of SEQ ID NOs: 34, 35, and 36, respectively, and the VL comprises the light chain CDR1, CDR2, and CDR3 having the amino acid sequences of SEQ ID NOs: 38, 39, and 40, respectively; 6) the VH comprises the heavy chain CDR1, CDR2, and CDR3 sequences having the amino acid sequences of SEQ ID NOs: 42, 43, and 44, respectively, and the VL comprises the light chain CDR1, CDR2, and CDR3 having the amino acid sequences of SEQ ID NOs:46, 47, and 48, respectively; 7) the VH comprises the heavy chain CDR1, CDR2, and CDR3 sequences having the amino acid sequences of SEQ ID NOs: 50, 51, and 52, respectively, and the VL comprises the light chain CDR1, CDR2, and CDR3 having the amino acid sequences of SEQ ID NOs: 54, 55, and 56, respectively; 8) the VH comprises the heavy chain CDR1, CDR2, and CDR3 sequences having the amino acid sequences of SEQ ID NOs: 58, 59, and 60, respectively, and the VL comprises the light chain CDR1, CDR2, and CDR3 having the amino acid sequences of SEQ ID NOs: 62, 63, and 64, respectively; 9) the VH comprises the heavy chain CDR1, CDR2, and CDR3 sequences having the amino acid sequences of SEQ ID NOs: 66, 67, and 68, respectively, and the VL comprises the light chain CDR1, CDR2, and CDR3 having the amino acid sequences of SEQ ID NOs:70, 71, and 72, respectively; or 10) the VH comprises the heavy chain CDR1, CDR2, and CDR3 sequences having the amino acid sequences of SEQ ID NOs: 74, 75, and 76, respectively, and the VL comprises the light chain CDR1, CDR2, and CDR3 having the amino acid sequences of SEQ ID NOs:78, 79, and 80, respectively.

In some embodiments, the VH comprises an amino acid sequence that is at least 90% identical to a sequence selected from the group consisting of SEQ ID NOs:1, 9, 17, 25, 33, 41, 49, 57, 65, and 73, and the VL comprises an amino acid sequence that is at least 90% identical to a sequence selected from the group consisting of SEQ ID NOs: 5, 13, 21, 29, 37, 45, 53, 61, 69, and 77, respectively.

In some embodiments, the VH comprises the amino acid sequence selected from the group consisting of SEQ ID NOs:1, 9, 17, 25, 33, 41, 49, 57, 65, and 73, or a variant thereof comprising up to about 3 amino acid substitutions in the VH; and the VL comprises the amino acid sequence selected from the group consisting of SEQ ID NOs:5, 13, 21, 29, 37, 45, 53, 61, 69, and 77, or a variant thereof comprising up to about 3 amino acid substitutions in the VL.

In some embodiments, in the isolated antibody or an antigen-binding fragment thereof, 1) the VH comprises an amino acid sequence of SEQ ID NO: 1, and the VL comprises an amino acid sequence of SEQ ID NO: 5; 2) the VH comprises an amino acid sequence of SEQ ID NO: 9, and the VL comprises an amino acid sequence of SEQ ID NO: 13; 3) the VH comprises an amino acid sequence of SEQ ID NO: 17, and the VL comprises an amino acid sequence of SEQ ID NO: 21; 4) the VH comprises an amino acid sequence of SEQ ID NO: 25, and the VL comprises an amino acid sequence of SEQ ID NO: 29; 5) the VH comprises an amino acid sequence of SEQ ID NO: 33, and the VL comprises an amino acid sequence of SEQ ID NO: 37; 6) the VH comprises an amino acid sequence of SEQ ID NO: 41, and the VL comprises an amino acid sequence of SEQ ID NO: 45; 7) the VH comprises an amino acid sequence of SEQ ID NO: 49, and the VL comprises an amino acid sequence of SEQ ID NO: 53; 8) the VH comprises an amino acid sequence of SEQ ID NO: 57, and the VL comprises an amino acid sequence of SEQ ID NO:61; 9) the VH comprises an amino acid sequence of SEQ ID NO: 65, and the VL comprises an amino acid sequence of SEQ ID NO: 69; or 10) the VH comprises an amino acid sequence of SEQ ID NO: 73, and the VL comprises an amino acid sequence of SEQ ID NO: 77.

In some embodiments, the EC₅₀ of the binding between the isolated antibody or antigen-binding fragment thereof and a cell expressing the B7-H3 is 10⁻⁷ M to about 10⁻¹² M, preferably about 10⁻⁸ M to about 10⁻¹² M, more preferably about 10⁻⁹ M to about 10⁻¹² M.

In some embodiments, the binding of the isolated antibody or antigen-binding fragment thereof to a cell expressing the B7-H3 has an EC₅₀ lower than that of the reference antibody MGA271.

In some embodiments, the antibody is a mouse, chimeric, humanized or human antibody.

In some embodiments, the humanized antibody comprises a VH comprising an amino acid sequence selected from the group consisting of SEQ ID NOs: 81-87, and a VL comprising an amino acid sequence selected from the group consisting of SEQ ID NOs: 88-91.

In some embodiments, the humanized antibody comprises: 1) a VH comprising an amino acid sequence of SEQ ID NO: 81, and a VL comprising an amino acid sequence of SEQ ID NO: 88; 2) a VH comprising an amino acid sequence of SEQ ID NO: 82, and a VL comprising an amino acid sequence of SEQ ID NO: 88; 3) a VH comprising an amino acid sequence of SEQ ID NO: 83, and a VL comprising an amino acid sequence of SEQ ID NO: 89; 4) a VH comprising an amino acid sequence of SEQ ID NO: 84, and a VL comprising an amino acid sequence of SEQ ID NO: 89; 5) a VH comprising an amino acid sequence of SEQ ID NO: 85, and a VL comprising an amino acid sequence of SEQ ID NO: 90; 6) a VH comprising an amino acid sequence of SEQ ID NO: 86, and a VL comprising an amino acid sequence of SEQ ID NO: 91; or 7) a VH comprising an amino acid sequence of SEQ ID NO: 87, and a VL comprising an amino acid sequence of SEQ ID NO: 91.

In some embodiments, the VH is fused to a heavy chain constant region of an immunoglobulin.

In some embodiments, the heavy chain constant region is from human IgG1.

In some embodiments, the heavy chain constant region comprises a modification that enhances the ADCC effect of the antibody.

In some embodiments, the heavy chain constant region comprises mutations K214R, L235V, F243L, R292P, Y300L, D356E, L358M and P396L.

In some embodiments, the heavy chain constant region comprises an amino acid sequence of SEQ ID NO: 93 or 94.

In some embodiments, the VL is fused to a light chain constant region (CL) of an immunoglobulin.

In some embodiments, the light chain constant region comprises an amino acid sequence of SEQ ID NO: 92.

In some embodiments, the isolated antibody or antigen-binding fragment thereof is conjugated to a toxin or a chemotherapeutic agent.

In some embodiments, the toxin is a Pseudomonas exotoxin (PE), ricin, abrin, diphtheria toxin, ribotoxin, saporin, calicheamicin, or a botulinum toxin.

In some embodiments, the chemotherapeutic agent is Monomethyl Auristatin E or a maytansinoid.

In another aspect, the present disclosure provides a bispecific antibody comprising the isolated antibody or an antigen-binding fragment and a second antibody moiety.

In some embodiments, the second antibody moiety is able to specifically bind to an antigen other than B7-H3.

In some embodiments, the antigen other than the B7-H3 is CTLA-4, PD-L1, TIM-3, or LAG-3.

In some embodiments, the second antibody moiety is a Fab, a Fab′, a (Fab′)₂, an Fv, a single chain Fv (scFv), an scFv-scFv, a minibody, a diabody, an sdAb, or an antibody mimetic.

In another aspect, the present disclosure provides a pharmaceutical composition for use in treating a cancer comprising: the isolated antibody or antigen-binding fragment thereof or the bispecific antibody; and a pharmaceutically acceptable carrier.

In some embodiments, the pharmaceutical composition further comprises one or more other anti-cancer agents.

In some embodiments, the cancer is a B7-H3-expressing cancer; preferably NSCLC, HNSCC, or breast cancer.

In another aspect, the present disclosure provides a method of treating cancer, which comprises administrating to a subject in need thereof a therapeutically effective amount of the isolated antibody or antigen-binding fragment thereof, the bispecific antibody or the pharmaceutical composition.

In some embodiments, the cancer is a B7-H3-expressing cancer; preferably NSCLC, HNSCC, or breast cancer.

In another aspect, the present disclosure provides uses of the isolated antibody or antigen-binding fragment thereof or the bispecific antibody in the manufacture of a medicament for the treatment of a cancer.

In some embodiments, the cancer is a B7-H3-expressing cancer; preferably NSCLC, HNSCC, or breast cancer.

In another aspect, the present disclosure provides the isolated antibody or antigen-binding fragment thereof, the bispecific antibody or the pharmaceutical composition for use in treating a cancer in a subject in need thereof.

In some embodiments, the cancer is a B7-H3-expressing cancer; preferably NSCLC, HNSCC, or breast cancer.

In some embodiments, the isolated antibody or antigen-binding fragment, the bispecific antibody or pharmaceutical composition is for systemic or local administration.

In some embodiments, the isolated antibody or antigen-binding fragment, the bispecific antibody or pharmaceutical composition is for intravenous administration or intratumoral administration.

The antibodies provided herein are able to kill tumors such as NSCLC and HNSCC dependent on ADCC and CDC, and have better efficacy than positive control MGA271 (Enoblituzumab) and DS-5573a.

### BRIEF DESCRIPTION OF THE DRAWINGS

FIG. 1. Results of mouse mAb FACS binding analysis.

FIG. 2. Results of chimeric antibody CDC induction analysis.

FIG. 3. Results of chimeric antibody ADCC induction analysis.

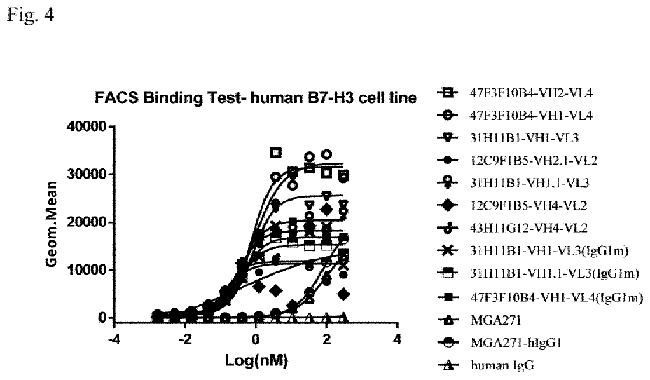

FIG. 4. Results of humanized antibody FACS binding analysis.

FIG. 5. Results of humanized antibody CDC induction analysis.

FIGS. 6A-6B. Results of humanized antibody ADCC induction analysis.

### DETAILED DESCRIPTION

The present disclosure provides the anti-B7-H3 monoclonal antibodies and their applications. The disclosure pertains to the amino acid sequences of the heavy chain variable domains (VH) and the light chain variable domains (V_{L}) of the mouse anti-B7-H3 monoclonal antibodies clones, 12C9F1B5,38F11B2, 34A9C12, 37D11F5B8, 37F11A10, 43H11G12, 47F3F10B4, 47C7E3, 31H11B1, and 38D6A4. It also pertains to the amino acid sequences of the heavy chain variable domains (VH) and the light chain variable domains (V_{L}) after the humanization or post-translational modification on some of these mouse anti-B7-H3 monoclonal antibodies clones. The disclosure pertains to methods of the generation of the anti-B7-H3 monoclonal antibodies.

The present disclosure provides the chimeric anti-B7-H3 monoclonal antibodies by fusing variable domains of the heavy and light chains of the disclosed clones with the constant region of human IgG1. The present disclosure provides humanized forms of the heavy chain variable domains (VH) and the light chain variable domains (V_{L}) of the mouse anti-B7-H3 monoclonal antibodies clones. The humanized anti-B7-H3 monoclonal antibodies were generated by fusing the humanized variable domains of the heavy and light chains of the disclosed clones with the constant region of human IgG1.

### I. Definitions

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al, Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment.

The term “effective amount” used herein refers to an amount of an agent or a combination of agents, sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition can: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “antibody,” “antibody moiety” or “antibody construct” is used in the broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs” (Variable domain of the heavy chain of the Heavy chain antibody). Some VHHs can also be known as nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

An “isolated” antibody is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and can include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody includes the antibody in situ within recombinant cells since at least one component of the antibody's natural environment will not be present. Ordinarily, however, an isolated polypeptide, antibody, or construct will be prepared by at least one purification step.

The “variable region” or “variable domain” of an antibody refers to the amino-terminal domains of the heavy or light chain of the antibody. The variable domains of the heavy chain and light chain can be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”. V_{H}H is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, deamidations) that can be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the application can be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Nat'l. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody,” “intact antibody,” or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain (such as V_{H}H) and an Fc region. The constant domains can be native sequence constant domains (e.g., human native sequence constant domains) or amino acid sequence variants thereof. In some cases, the intact antibody can have one or more effector functions.

An “antibody fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “constant domain region” or “constant region” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or CL) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies described herein comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l. Acad. Sci. USA, 81:6851-6855 (1984)). “Humanized antibody” is used as a subset of “chimeric antibodies”.

“Humanized” forms of non-human (e.g., llama or camelid) antibodies are antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs, and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs can comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the V_{H}. The variable domain residues are numbered according to Kabat et al., supra, for each of these definitions.

The amino acid residues of a single-domain antibody (such as V_{H}H) are numbered according to the general numbering for V_{H} domains given by Kabat et al. (“Sequence of proteins of immunological interest”, US Public Health Services, NIH Bethesda, Md., Publication No. 91), as applied to V_{H}H domains from Camelids in the article of Riechmann and Muyldermans, J. Immunol. Methods 2000 Jun. 23; 240 (1-2): 185-195. According to this numbering, FR1 of a V_{H}H comprises the amino acid residues at positions 1-30, CDR1 of a V_{H}H comprises the amino acid residues at positions 31-35, FR2 of a V_{H}H comprises the amino acids at positions 36-49, CDR2 of a V_{H}H comprises the amino acid residues at positions 50-65, FR3 of a V_{H}H comprises the amino acid residues at positions 66-94, CDR3 of a V_{H}H comprises the amino acid residues at positions 95-102, and FR4 of a V_{H}H comprises the amino acid residues at positions 103-113. In this respect, it should be noted that—as is well known in the art for V_{H} domains and for V_{H}H domains—the total number of amino acid residues in each of the CDRs can vary and cannot correspond to the total number of amino acid residues indicated by the Kabat numbering (that is, one or more positions according to the Kabat numbering cannot be occupied in the actual sequence, or the actual sequence can contain more amino acid residues than the number allowed for by the Kabat numbering).

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence can contain fewer or additional amino acids corresponding to a shortening of, or insertion into, a FR or HVR of the variable domain. For example, a heavy-chain variable domain can include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues can be determined for a given antibody by alignment at regions of homology of the sequence of the antibody with a “standard” Kabat numbered sequence.

In certain embodiments, an antibody can also be analyzed using the IMGT information system. The IMGT information system was created in 1989 by Marie-Paule Lefranc, and now is used as a global reference in immunogenetics and immunoinformatics (see, e.g, Lefranc M-P, The Immunologist 7: 132-136 (1999) and Lefranc M-P et al., Nucleic Acids Res 27: 209-212 (1999)). The IMGT numbering scheme is summarized in Table 2 (see, https://www.imgt.org/IMGTinformation/).

TABLE 2
IMGT numbering scheme

| | | | | | | For germline | For rearranged | |
|---|---|---|---|---|---|---|---|---|
| | | | | | | V-GENE (*) | V-J-GENEs and | |
| | | | | | | CDR3-IMGT | V-D-J-GENEs | |
| | FR1- | CDR1- | FR2- | CDR2- | FR3- | Germline | Rearranged | FR4- |
| | IMGT | IMGT | IMGT | IMGT | IMGT | CDR3-IMGT | CDR3-IMGT | IMGT |
| Amino | 1 --> 26 | 27 --> 38 | 39 --> 55 | 56 --> 65 | 66 --> | 105 --> 116 | 105 --> 117 | 118 --> |
| acid | (C 23) | | (W 41) | | 104 | | (112.1, 111.1, | 129 |
| numbering | | | | | (C 104) | | 112.2, 111.2, etc.) | |
| Number of | 25-26 aa | 5-12 aa | 16-17 aa | 0-10 aa | 36-39 aa | 2-12 aa | 2-13 aa | 10-12 aa |
| amino | | | | | | | (more than 13 aa) | |
| acids | | | | | | | | |

(*) + 0, 1 or 2 nucleotide(s) before the heptamer.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the V_{L}, the subgroup can be subgroup kappa I, kappa II, kappa III or kappa IV as in Kabat et al., supra. Additionally, for the V_{H}, the subgroup can be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework can comprise the same amino acid sequence thereof, or it can contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “affinity-matured” antibody is one with one or more alterations in one or more CDRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by V_{H}- and V_{L}-domain shuffling. Random mutagenesis of CDR and/or framework residues is described by, for example: Barbas et al. Proc Nat'l. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As use herein, the term “specifically binds,” “specifically recognizes,” or is “specific for” refers to measurable and reproducible interactions such as binding between a target and an antigen binding protein (such as a mAb), which is determinative of the presence of the target in the presence of a heterogeneous population of molecules including biological molecules. For example, an antigen binding protein (such as a mAb) that specifically binds a target (which can be an epitope) is an antigen binding protein (such as a mAb) that binds this target with greater affinity, avidity, more readily, and/or with greater duration than it binds other targets. In some embodiments, the extent of binding of an antigen binding protein (such as a mAb) to an unrelated target is less than about 10% of the binding of the antigen binding protein (such as a mAb) to the target as measured, e.g., by a radioimmunoassay (RIA). In some embodiments, an antigen binding protein (such as a mAb) that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antigen binding protein specifically binds an epitope on a protein that is conserved among the protein from different species. In some embodiments, specific binding can include, but does not require, exclusive binding.

The term “specificity” refers to selective recognition of an antigen binding protein (such as a mAb) for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. Unless otherwise indicated, the order in which the antigens bound by a bispecific antibody listed is arbitrary. That is, for example, the terms “anti-B7-H3/PD-L1,” “anti-PD-L1/B7-H3,” “B7-H3×PD-L1,” “PD-L1×B7-H3,” “PD-L1-B7-H3,” and “B7-H3-PD-L1” can be used interchangeably to refer to bispecific antibodies that specifically bind to both B7-H3 and PD-L1. The term “monospecific” as used herein denotes an antigen binding protein (such as a mAb) that has one or more binding sites each of which bind the same epitope of the same antigen.

The term “valent” as used herein denotes the presence of a specified number of binding sites in an antigen binding protein. A natural antibody for example or a full length antibody has two binding sites and is bivalent. As such, the terms “trivalent,” “tetravalent,” “pentavalent,” and “hexavalent” denote the presence of two binding site, three binding sites, four binding sites, five binding sites, and six binding sites, respectively, in an antigen binding protein.

“Antibody effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effector-less mutation.” In one aspect, the effector-less mutation comprises an N297A or DANA mutation (D265A and/or N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fe expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and natural killer (NK) cells. Alternatively, or additionally, ADCC activity of the molecule of interest can be assessed in vivo, e.g., in an animal model such as that disclosed in Clynes et al., Proc. Nat'l. Acad. Sci. USA 95:652-656 (1998).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region can be removed, for example, during production or purification of the antibody, or by recombinantly engineering the nucleic acid encoding a heavy chain of the antibody. Accordingly, a composition of intact antibodies can comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), can be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair. Binding affinity can be indicated by K_{D}, K_{off}, Kₒₙ, or Kₐ. The term “K_{off}”, as used herein, is intended to refer to the off rate constant for dissociation of an antibody (or antigen-binding domain) from the antibody/antigen complex, as determined from a kinetic selection set up, expressed in units of s⁻¹. The term “Kₒₙ.”, as used herein, is intended to refer to the on rate constant for association of an antibody (or antigen-binding domain) to the antigen to form the antibody/antigen complex, expressed in units of M⁻¹s⁻¹. The term equilibrium dissociation constant “K_{D}”, as used herein, refers to the dissociation constant of a particular antibody-antigen interaction, and describes the concentration of antigen required to occupy one half of all of the antibody-binding domains present in a solution of antibody molecules at equilibrium, and is equal to K_{off}/Kₒₙ, expressed in units of M. The measurement of K_{D} presupposes that all binding agents are in solution. In the case where the antibody is tethered to a cell wall, e.g., in a yeast expression system, the corresponding equilibrium rate constant is expressed as EC₅₀, which gives a good approximation of K_{D}. The affinity constant, Kₐ, is the inverse of the dissociation constant, K_{D}, expressed in units of M⁻¹.

The dissociation constant (K_{D}) is used as an indicator showing affinity of antibodies to antigens. For example, easy analysis is possible by the Scatchard method using antibodies marked with a variety of marker agents, as well as by using BiacoreX (made by Amersham Biosciences), which is an over-the-counter, measuring kit, or similar kit, according to the user's manual and experiment operation method attached with the kit. The K_{D} value that can be derived using these methods is expressed in units of M (moles per liter). An antibody or antigen-binding fragment thereof that specifically binds to a target can have a dissociation constant (K_{D}) of, for example, ≤10⁻¹ M, ≤10⁻⁶ M, ≤10⁻⁷ M, ≤10⁻¹ M, ≤10⁻⁹ M, ≤10⁻¹⁰ M, 10⁻¹¹ M, or 10⁻¹² M.

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

Half maximal inhibitory concentration (IC₅₀) is a measure of the effectiveness of a substance (such as an antibody) in inhibiting a specific biological or biochemical function. It indicates how much of a particular drug or other substance (inhibitor, such as an antibody) is needed to inhibit a given biological process (e.g., the binding between PD-L1 and B7-1, or component of a process, i.e., an enzyme, cell, cell receptor or microorganism) by half. The values are typically expressed as molar concentration. IC₅₀ is comparable to an EC₅₀ for agonist drug or other substance (such as an antibody). EC₅₀ also represents the plasma concentration required for obtaining 50% of a maximum effect in vivo. As used herein, an “IC₅₀” is used to indicate the effective concentration of an antibody (such as an anti-PD-L1 mAb) needed to neutralize 50% of the antigen bioactivity (such as PD-L1 bioactivity) in vitro. IC₅₀ or EC₅₀ can be measured by bioassays such as inhibition of ligand binding by FACS analysis (competition binding assay), cell based cytokine release assay, or amplified luminescent proximity homogeneous assay (AlphaLISA).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny cannot be completely identical in nucleic acid content to a parent cell, but can contain mutations. Mutant progeny that has the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X.”

The term “about X-Y” used herein has the same meaning as “about X to about Y.”

### H. Anti-B7-H3 Construct

### Anti-B7-H3 Monoclonal Antibody

An isolated anti-B7-H3 construct described herein comprises a monoclonal antibody (mAb) moiety that specifically recognizes or binds to B7-H3 (or “anti-B7-H3 mAb”). In some embodiments of the invention, an isolated anti-B7-H3 construct is a full-length IgG.

### B7 Superfamily and B7-H3

As used herein, the term “B7-H3” refers to a member of the human B7 family of proteins, a type I membrane protein with Ig-like domains also known as CD276.

Investigations into the ligands of the CD28 receptor have led to the characterization of a set of related molecules known as the B7 Superfamily. B7 family members are immunoglobulin superfamily members with an immunoglobulin-V-like and an immunoglobulin-C-like domain. The IgV and IgC domains of B7-family members are each encoded by single exons, with additional exons encoding leader sequences, transmembrane and cytoplasmic domains. The cytoplasmic domains are short, ranging in length from 19 to 62 amino-acid residues and can be encoded by multiple exons. B7-H3 is unique in that the major human form contains two extracellular tandem IgV-IgC domains (i.e., IgV-IgC-IgV-IgC) (Collins, M. et al. (2005), Genome Biol. 6:223.1-223.7). Members of the B7 family are predicted to form back-to-back, non-covalent homodimers at the cell surface, and such dimers have been found with respect to B7-1 (CD80) and B7-2 (CD86).

Although initially thought to comprise only 2 Ig domains (IgV-IgC) (Chapoval, A. et al. (2001), Nature Immunol. 2:269-274; Sun, M. et al. (2002), J. Immunol. 168:6294-6297), a four immunoglobulin extracellular domain variant (“4Ig-B7-H3”) has been identified and found to be more common human form of the protein (Sharpe, A. H. et al. (2002), Nature Rev. Immunol. 2:116-126). No functional difference has been observed between these two forms, since the natural murine form (2Ig) and the human 4Ig form exhibit similar function (Hofmeyer, K. et al. (2008), Proc. Natl. Acad. Sci. USA, 105(30):10277-10278). The 4Ig-B7-H3 molecule inhibits the natural killer cell-mediated lysis of cancer cells (Castriconi, R. et al., Proc. Natl. Acad. Sci. USA, 101(34): 12640-12645). The human B7-H3 (2Ig form) has been found to promote T-cell activation and IFN-gamma production by binding to a putative receptor on activated T cells (Chapoval, A. et al. (2001), Nature Immunol. 2:269-274; Xu, H. et al. (2009), Cancer Res. 69(15):5275-6281). Both B7-H4 and B7-H1 are potent inhibitors of immune function when expressed on tumor cells (Flies, D. B. et al. (2007), J. Immunother. 30(3):251-260).

The mode of action of B7-H3 is complex, as the protein mediates both T cell co-stimulation and co-inhibition (Hofmeyer, K. et al. (2008), Proc. Natl. Acad. Sci. USA, 105(30):10277-10278; Martin-Orozco, N. et al. (2007), Semin. Cancer Biol. 17(4):288-298; Subudhi, S. K. et al. (2005), J. Mol. Med. 83:193-202). B7-H3 binds to (TREM)-like transcript 2 (TLT-2) and co-stimulates T cell activation, but also binds to as yet unidentified receptor(s) to mediate co-inhibition of T cells. In addition, B7-H3, through interactions with unknown receptor(s) is an inhibitor for natural killer cells and osteoblastic cells (Hofmeyer, K. et al. (2008), Proc. Natl. Acad. Sci. USA, 105(30):10277-10278). The inhibition may operate through interactions with members of the major signaling pathways through which T cell receptor (TCR) regulates gene transcription (e.g., NFTA, NF-.kappa.B, or AP-1 factors).

B7-H3 co-stimulates CD4+ and CD8+ T-cell proliferation. B7-H3 also stimulates IFN-.gamma. production and CD8+ lytic activity (Chapoval, A. et al. (2001), Nature Immunol. 2:269-274; Sharpe, A. H. et al. (2002), Nature Rev. Immunol. 2:116-126). However, the protein also possibly acts through NFAT (nuclear factor for activated T cells), NF-.kappa.B (nuclear factor kappa B), and AP-1 (Activator Protein-1) factors to inhibit T-cell activation (Yi. K. H. et al. (2009), Immunol. Rev. 229:145-151). B7-H3 is also believed to inhibit Th1, Th2, or Th17 in vivo (Prasad, D. V. et al. (2004), J. Immunol. 173:2500-2506; Fukushima, A. et al. (2007), Immunol. Lett. 113:52-57; Yi. K. H. et al. (2009), Immunol. Rev. 229:145-151). Several independent studies have shown that human malignant tumor cells exhibit a marked increase in expression of B7-H3 protein and that this increased expression was associated with increased disease severity (Zang, X. et al. (2007), Clin. Cancer Res. 13:5271-5279), suggesting that B7-H3 is exploited by tumors as an immune evasion pathway (Hofmeyer, K. et al. (2008), Proc. Natl. Acad. Sci. (U.S.A.) 105(30):10277-10278).

Molecules that block the ability of a B7 molecule to bind to a T-cell receptor (e.g., CD28) inhibit the immune system and have been proposed as treatments for autoimmune disease (Linsley, P. S. et al. (2009), Immunolog. Rev. 229:307-321). Neuroblastoma cells expressing 4Ig-B7-H3 treated with anti-4Ig-B7-H3 antibodies were more susceptible to NK cells. However, it is unclear whether this activity can be attributed to only antibodies against the 4Ig-B7-H3 form because all reported antibodies raised against the 4Ig-B7-H3 also bound the two Ig-like form of B7-H3 (Steinberger, P. et al. (2004), J. Immunol. 172(4): 2352-2359 and Castriconi et al. (2004), Proc. Natl. Acad. Sci. USA, 101(34):12640-12645).

B7-H3 is not expressed on resting B or T cells, monocytes, or dendritic cells, but it is induced on dendritic cells by IFN-gamma and on monocytes by GM-CSF (Sharpe, A. H. et al. (2002), Nature Rev. Immunol. 2:116-126). The receptor(s) that bind B7-H3 have not been fully characterized. Early work suggested one such receptor would need to be rapidly and transiently up-regulated on T cells after activation (Loke, P. et al. (2004), Arthritis Res. Ther. 6:208-214).

In addition to its expression on neuroblastoma cells, human B7-H3 is also known to be expressed on a variety of other cancer cells (e.g., gastric, ovarian and non-small cell lung cancers). B7-H3 protein expression has been immunohistologically detected in tumor cell lines (Chapoval, A. et al. (2001), Nature Immunol. 2:269-274; Saatian, B. et al. (2004), Amer. J. Physiol. Lung Cell. Mol. Physiol. 287:L217-L225; Castriconi et al. (2004), Proc. Natl. Acad. Sci. USA, 101(34):12640-12645); Sun, M. et al. (2002), J. Immunol. 168:6294-6297). mRNA expression has been found in heart, kidney, testes, lung, liver, pancreas, prostate, colon, and osteoblast cells (Collins, M. et al. (2005), Genome Biol. 6:223.1-223.7). At the protein level, B7-H3 is found in human liver, lung, bladder, testis, prostate, breast, placenta, and lymphoid organs (Hofmeyer, K. et al. (2008), Proc. Natl. Acad. Sci. USA, 105(30):10277-10278).

In some embodiments, an anti-B7-H3 mAb of the present disclosure can cross-react with B7-H3 from species other than human, or other proteins which are structurally related to human B7-H3. In some embodiments, an anti-B7-H3 mAb of the application is completely specific for human B7-H3 and not exhibit species or other types of cross-reactivity.

### Antibody Affinity

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

In some embodiments, the K_{D} of the binding between the anti-B7-H3 mAb and B7-H3 is about 10⁻⁵ M to about 10⁻⁶ M, about 10⁻⁶ M to about 10⁻⁷ M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁵ M to about 10⁻¹² M, about 10⁻⁶ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁵ M to about 10⁻¹¹ M, about 10⁻⁷ M to about 10⁻¹¹ M, about 10⁻¹ M to about 10⁻¹¹ M, about 10⁻⁹ M to about 10⁻¹¹ M, about 10⁻⁵ M to about 10⁻¹⁰ M, about 10⁻⁷ M to about 10⁻¹⁰ M, about 10⁻⁸ M to about 10⁻¹⁰ M, about 10⁻⁵ M to about 10⁻⁹ M, about 10⁻⁷ M to about 10⁻⁹ M, about 10⁻⁵ M to about 10⁻⁸ M, or about 10⁻⁶ M to about 10⁻⁸ M.

In some embodiments, the Kₒₙ of the binding between the anti-B7-H3 mAb and B7-H3 is about 10² M⁻¹s⁻¹ to about 10⁴ M⁻¹s⁻¹, about 10⁴ M⁻¹s⁻¹ to about 106 M⁻¹s⁻¹, about 106 M⁻¹s⁻¹ to about 10⁷ M⁻¹s⁻¹, about 102 M⁻¹s⁻¹ to about 10⁷ M⁻¹s⁻¹, about 10³ M^{-IS-}1 to about 10⁷ M⁻¹s⁻¹, about 10⁴ M⁻¹s⁻¹ to about 10⁷ M⁻¹s⁻¹, about 10⁵ M⁻¹s⁻¹ to about 10⁷ M⁻¹s⁻¹, about 10³ M^{-IS-}1 to about 106 M⁻¹s⁻¹, or about 10⁴ M⁻¹s⁻¹ to about 10⁶ M⁻¹s⁻¹.

In some embodiments, the K_{off} of the binding between the anti-B7-H3 mAb and B7-H3 is about 1 s⁻¹ to about 10⁻² s⁻¹, about 10⁻² s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁶ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s-t.

In some embodiments, the E₅₀ of the anti-B7-H3 mAb is less than 10 nM in a FACS study of the binding of the anti-B7-H3 mAb to B7-H3-expressing CHO-K1 cells. In some embodiments, the EC₅₀ of the anti-B7-H3 mAb is less than 1 nM.

### Chimeric or Humanized Antibodies

In some embodiments, the anti-B7-H3 antibody provided herein is a chimeric antibody. Certain chimeric antibodies are described, e.g., in U.S. Pat. No. 4,816,567; and Morrison et al., Proc. Nat'l. Acad. Sci. USA, 81:6851-6855 (1984)). In one example, achimeric antibody comprises a non-human variable region (e.g., a variable region derived from a camelid species, such as llama) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); US Pat. Nos. 5, 821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nat'l. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the mAbs are modified, such as humanized, without diminishing the native affinity of the domain for antigen and while reducing its immunogenicity with respect to a heterologous species. For example, the amino acid residues of the antibody heavy chain and light chain variable domains (V_{H} and VL) can be determined, and one or more of the mouse amino acids, for example, in the framework regions, are replaced by their human counterpart as found in the human consensus sequence, without that polypeptide losing its typical character, i.e., the humanization does not significantly affect the antigen binding capacity of the resulting polypeptide. Humanization of mouse monoclonal antibodies requires the introduction and mutagenesis of a limited amount of amino acids in two chains, the light and the heavy chain and the preservation of the assembly of both chains.

### Human Antibodies

In some embodiments, the anti-B7-H3 antibody, particularly mAb, provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008). Transgenic mice or rats capable of producing fully human single-domain antibodies are known in the art. See, e.g., US20090307787A1, U.S. Pat. No. 8,754,287, US20150289489A1, US20100122358A1, and WO2004049794.

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Nat'l. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies can also be generated by isolating Fv clone variable domain sequences selected from human-derived phage display libraries. Such variable domain sequences can then be combined with a desired human constant domain. Techniques for selecting human antibodies from antibody libraries are described below.

### Library-Derived Antibodies

Antibodies of the present application can be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Nat'l. Acad. Sci. USA 101(34): 12467-12472 (2004); and Lee et al., J. Immunol. Methods 284(1-2): 119-132(2004). Methods for constructing single-domain antibody libraries have been described, for example, see U.S. Pat. No. 7,371,849.

In certain phage display methods, repertoires of V_{H} and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Anti-B7-H3 Antibodies

In some embodiments, there is provided an anti-B7-H3 mAb comprising a heavy chain variable domain (V_{H}) with a heavy chain CDR1 comprising the amino acid sequence of any one of SEQ ID NO: 2 (DHYMH), SEQ ID NO: 10 (GMN), SEQ ID NO: 18 (GIN), SEQ ID NO: 26 (DFGMN), SEQ ID NO: 34 (NNGMN), SEQ ID NO: 42 (NYGMN), SEQ ID NO: 50 (DFGMN), SEQ ID NO: 58 (DYGMN), SEQ ID NO: 66 (NFGMN), and SEQ ID NO: 74 (SYWMH), or a variant thereof comprising up to about 3 (such as about any of 1, 2, or 3) amino acid substitutions (for a CDR with 3 amino acid residues, their variants may comprise up to about 2 (such as 1 or 2) amino acid substitutions); a heavy chain CDR2 comprising the amino acid sequence of any one of

SEQ ID NO: 3
(YIFPNNGVSGYNQNFKD),
SEQ ID NO: 11
(WINPYSGVPTYADDFKG),
SEQ ID NO: 19
(WINPYSGVPTYADDFKG),
SEQ ID NO: 27
(WINPYSGVPTYADEFKG),
SEQ ID NO: 35
(WINPYSGVPTYADDFKG),
SEQ ID NO: 43
(WINPYSGVPTYADDFKG),
SEQ ID NO: 51
(WLNSYSGVPTYADDFKG),
SEQ ID NO: 59
(WINIYSGVPTYADDFKG),
SEQ ID NO: 67
(WIYTPSGVPIYADDFKG),
and
SEQ ID NO: 75
(HINPNSGSTNQNAKFKN),

or a variant thereof comprising up to about 3 (such as about any of 1, 2, or 3) amino acid substitutions; and a heavy CDR3 comprising the amino acid sequence of any one of

SEQ ID NO: 4
(RQLGLGTMDY),
SEQ ID NO: 12
(DDGFHYTMDY),
SEQ ID NO: 20
(DDGFHYTMDY),
SEQ ID NO: 28
(DDGYHYTMDY),
SEQ ID NO: 36
(DDGYHYTMDF),
SEQ ID NO: 44
(DDGFHYTMDY),
SEQ ID NO: 52
(DDGYNYTMNY),
SEQ ID NO: 60
(DDGYHYTMNY),
SEQ ID NO: 68
(DDGYYYTMDY),
and
SEQ ID NO: 76
(SGSNYRRNYFDY),

or a variant thereof comprising up to about 3 (such as about any of 1, 2, or 3) amino acid substitutions; and a light chain variable domain (VL) with a light chain CDR1 comprising the amino acid sequence of any one of

SEQ ID NO: 6
(RASKSVSTSGYNYMH),
SEQ ID NO: 14
(RSSQNLVHSNGITYLH),
SEQ ID NO: 22
(RSSQNLVHSNGITYLH),
SEQ ID NO: 30
(RSSQSLIHSNGITYLH),
SEQ ID NO: 38
(RSSQSLVHSNGITYLH),
SEQ ID NO: 46
(RSSQSLLHSNGITYLH),
SEQ ID NO: 54
(RSSQSLVHSNGITYLH),
SEQ ID NO: 62
(RSSQSLVHSNGITYLH),
SEQ ID NO: 70
(RSSQSLVHSNGDTYLH),
and
SEQ ID NO: 78
(KASDHINNWLA),

or a variant thereof comprising up to about 3 (such as about any of 1, 2, or 3) amino acid substitutions; a light chain CDR2 comprising the amino acid sequence of any one of SEQ ID NO: 7 (LASNLES), SEQ ID NO: 15 (KVSNRFS), SEQ ID NO: 23 (KVSNRFS), SEQ ID NO: 31 (KVSNRFS), SEQ ID NO: 39 (KVSHRFS), SEQ ID NO: 47 (KVSHRFS), SEQ ID NO: 55 (KVSNRFS), SEQ ID NO: 63 (KVSNRFS), SEQ ID NO: 71 (KVSNRFS), and SEQ ID NO: 79 (GATSLET), or a variant thereof comprising up to about 3 (such as about any of 1, 2, or 3) amino acid substitutions; and a light chain CDR3 comprising the amino acid sequence of any one of

SEQ ID NO: 8
(QHSKELPWT),
SEQ ID NO: 16
(SQSTLVPWT),
SEQ ID NO: 24
(SQSTLVPWT),
SEQ ID NO: 32
(SQSTHVPWT),
SEQ ID NO: 40
(SQSTHVPWT),
SEQ ID NO: 48
(SQSTHVPWT),
SEQ ID NO: 56
(SQSTHVPWT),
SEQ ID NO: 64
(SQSTHVPWT),
SEQ ID NO: 72
(SQSTHVPWT),
and
SEQ ID NO: 80
(QQYWSTPLT),

or a variant thereof comprising up to about 3 (such as about any of 1, 2, or 3) amino acid substitutions. In some embodiments, the K_{D} of the binding between the anti-B7-H3 mAb and B7-H3 is about 10⁻⁵ M to about 10⁻¹² M (such as about 10⁻⁷ M to about 10⁻¹² M, or about 10⁻⁸ M to about 10⁻¹² M). In some embodiments, the anti-B7-H3 antibody is rodent, chimeric, human, partially humanized, or fully humanized.

In some embodiments, the anti-B7-H3 mAb comprises a V_{H} CDR3 comprising the amino acid sequence of any one of SEQ ID NOs: 4, 12, 20, 28, 36, 44, 52, 60, 68, and 76 and a VL CDR3 comprising the amino acid sequence of any one of SEQ ID NOs: 8, 16, 24, 32, 40, 48, 56, 64, 72, and 80, and the amino acid substitutions are in CDR1 and/or CDR2 of V_{H} and VL domains.

Thus, in some embodiments, there is provided an anti-B7-H3 mAb comprising a heavy chain variable domain (V_{H}) with a CDR1 comprising the amino acid sequence of any one of SEQ ID NOs: 2, 10, 18, 26, 34, 42, 50, 58, 66, and 74, or a variant thereof comprising up to about 3 (such as about any of 1, 2, or 3) amino acid substitutions; a CDR2 comprising the amino acid sequence of any one of SEQ ID NOs: 3, 11, 19, 27, 35, 43, 51, 59, 67, and 75, or a variant thereof comprising up to about 3 (such as about any of 1, 2, or 3) amino acid substitutions; and a CDR3 comprising the amino acid sequence of any one of SEQ ID NOs: 4, 12, 20, 28, 36, 44, 52, 60, 68, and 76; and a light chain variable domain (VL) with a CDR1 comprising the amino acid sequence of any one of SEQ ID NOs: 6, 14, 22, 30, 38, 46, 54, 62, 70, and 78, or a variant thereof comprising up to about 3 (such as about any of 1, 2, or 3) amino acid substitutions; a CDR2 comprising the amino acid sequence of any one of SEQ ID NOs: 7, 15, 23, 31, 39, 47, 55, 63, 71, and 79, or a variant thereof comprising up to about 3 (such as about any of 1, 2, or 3) amino acid substitutions; and a CDR3 comprising the amino acid sequence of any one of SEQ ID NOs: 8, 16, 24, 32, 40, 48, 56, 64, 72, and 80. In some embodiments, the K_{D} of the binding between the anti-B7-H3 mAb and B7-H3 is about 10⁻⁵ M to about 10⁻¹² M (such as about 10⁻⁷ M to about 10⁻¹² M, or about 10⁻⁸ M to about 10⁻¹² M), or less. In some embodiments, the anti-B7-H3 mAb is rodent, chimeric, human, partially humanized, or fully humanized.

In some embodiments, there is provided an anti-B7-H3 mAb comprising a heavy chain variable domain (V_{H}) with a CDR1 comprising an amino acid sequence of any one of SEQ ID NOs: 2, 10, 18, 26, 34, 42, 50, 58, 66, and 74; a CDR2 comprising an amino acid sequence of any one of SEQ ID NOs: 3, 11, 19, 27, 35, 43, 51, 59, 67, and 75; and a CDR3 comprising an amino acid sequence of any one of SEQ ID NOs: 4, 12, 20, 28, 36, 44, 52, 60, 68, and 76; and a light chain variable domain (VL) with a CDR1 comprising the amino acid sequence of any one of SEQ ID NOs: 6, 14, 22, 30, 38, 46, 54, 62, 70, and 78; a CDR2 comprising the amino acid sequence of any one of SEQ ID NOs: 7, 15, 23, 31, 39, 47, 55, 63, 71, and 79; and a CDR3 comprising the amino acid sequence of any one of SEQ ID NOs: 8, 16, 24, 32, 40, 48, 56, 64, 72, and 80. In some embodiments, the K_{D} of the binding between the anti-B7-H3 mAb and B7-H3 is about 10⁻⁵ M to about 10⁻¹² M (such as about 10⁻⁷ M to about 10⁻¹² M, or about 10⁻¹ M to about 10⁻¹² M). In some embodiments, the anti-B7-H3 mAb is rodent, chimeric, human, partially humanized, or fully humanized.

In some embodiments, there is provided an anti-B7-H3 mAb comprising: 1) a V_{H} comprising the heavy chain CDR1, CDR2, and CDR3 sequences having the amino acid sequences of SEQ ID NOs: 2, 3, and 4, respectively, and a VL comprises the light chain CDR1, CDR2, and CDR3 having the amino acid sequences of SEQ ID NOs: 6, 7, and 8, respectively; 2) a V_{H} comprising the heavy chain CDR1, CDR2, and CDR3 sequences having the amino acid sequences of SEQ ID NOs: 10, 11, and 12, respectively, and a VL comprising the light chain CDR1, CDR2, and CDR3 having the amino acid sequences of SEQ ID NOs: 14, 15, and 16, respectively; 3) a V_{H} comprising the heavy chain CDR1, CDR2, and CDR3 sequences having the amino acid sequences of SEQ ID NOs: 18, 19, and 20, respectively, and a VL comprising the light chain CDR1, CDR2, and CDR3 having the amino acid sequences of SEQ ID NOs: 22, 23, and 24, respectively; 4) a V_{H} comprises the heavy chain CDR1, CDR2, and CDR3 sequences having the amino acid sequences of SEQ ID NOs: 26, 27, and 28, respectively, and a VL comprising the light chain CDR1, CDR2, and CDR3 having the amino acid sequences of SEQ ID NOs: 30, 31, and 32, respectively; 5) a V_{H} comprising the heavy chain CDR1, CDR2, and CDR3 sequences having the amino acid sequences of SEQ ID NOs: 34, 35, and 36, respectively, and a VL comprising the light chain CDR1, CDR2, and CDR3 having the amino acid sequences of SEQ ID NOs: 38, 39, and 40, respectively; 6) a V_{H} comprising the heavy chain CDR1, CDR2, and CDR3 sequences having the amino acid sequences of SEQ ID NOs: 42, 43, and 44, respectively, and a VL comprising the light chain CDR1, CDR2, and CDR3 having the amino acid sequences of SEQ ID NOs:46, 47, and 48, respectively; 7) a V_{H} comprising the heavy chain CDR1, CDR2, and CDR3 sequences having the amino acid sequences of SEQ ID NOs: 50, 51, and 52, respectively, and a VL comprising the light chain CDR1, CDR2, and CDR3 having the amino acid sequences of SEQ ID NOs: 54, 55, and 56, respectively; 8) a V_{H} comprising the heavy chain CDR1, CDR2, and CDR3 sequences having the amino acid sequences of SEQ ID NOs: 58, 59, and 60, respectively, and a VL comprising the light chain CDR1, CDR2, and CDR3 having the amino acid sequences of SEQ ID NOs: 62, 63, and 64, respectively; 9) a V_{H} comprising the heavy chain CDR1, CDR2, and CDR3 sequences having the amino acid sequences of SEQ ID NOs: 66, 67, and 68, respectively, and a VL comprising the light chain CDR1, CDR2, and CDR3 having the amino acid sequences of SEQ ID NOs:70, 71, and 72, respectively; or 10) a V_{H} comprising the heavy chain CDR1, CDR2, and CDR3 sequences having the amino acid sequences of SEQ ID NOs: 74, 75, and 76, respectively, and a VL comprising the light chain CDR1, CDR2, and CDR3 having the amino acid sequences of SEQ ID NOs:78, 79, and 80, respectively.

In some embodiments, there is provided an anti-B7-H3 mAb comprising: 1) a V_{H} comprising an amino acid sequence of SEQ ID NO: 1, and a VL comprising an amino acid sequence of SEQ ID NO: 5 (47F3F10B4); 2) a V_{H} comprising an amino acid sequence of SEQ ID NO: 9, and a VL comprising an amino acid sequence of SEQ ID NO: 13 (12C9F1B5); 3) a V_{H} comprising an amino acid sequence of SEQ ID NO: 17, and a VL comprising an amino acid sequence of SEQ ID NO: 21 (34A9C12); 4) a V_{H} comprising an amino acid sequence of SEQ ID NO: 25, and a VL comprising an amino acid sequence of SEQ ID NO: 29 (38D6A4); 5) a V_{H} comprising an amino acid sequence of SEQ ID NO: 33, and a VL comprising an amino acid sequence of SEQ ID NO: 37 (37D11F5B8); 6) a V_{H} comprising an amino acid sequence of SEQ ID NO: 41, and a VL comprising an amino acid sequence of SEQ ID NO: 45 (37F11A10); 7) a V_{H} comprising an amino acid sequence of SEQ ID NO: 49, and a VL comprising an amino acid sequence of SEQ ID NO: 53 (43H11G12); 8) a V_{H} comprising an amino acid sequence of SEQ ID NO: 57, and a VL comprising an amino acid sequence of SEQ ID NO:61 (38F11B2); 9) a V_{H} comprising an amino acid sequence of SEQ ID NO: 65, and a VL comprising an amino acid sequence of SEQ ID NO: 69 (47C7E3); or 10) a V_{H} comprising an amino acid sequence of SEQ ID NO: 73, and a VL comprising an amino acid sequence of SEQ ID NO: 77 (31H11B1).

The CDRs can be combined in various pair-wise combinations to generate a number of humanized anti-B7-H3 antibodies. Humanized substitutions will be clear to those skilled in the art. For example, potentially useful humanizing substitutions can be determined by comparing the FR sequences of a naturally occurring V_{H} or VL with the corresponding FR sequences of one or more closely related human V_{H} or VL, then introducing one or more of such potentially useful humanizing substitutions into said V_{H} or VL using methods known in the art (also as described herein). The humanized heavy chains and light chains are paired. The resulting humanized antibodies can be tested for their B7-H3 binding affinity, for stability, for ease and level of expression, and/or for other desired properties. An anti-B7-H3 mAb described herein can be partially or fully humanized. Preferably, the resulting humanized antibody, such as humanized mAb, or an antigen binding fragment thereof, binds to B7-H3 with K_{D}, Kₒₙ, K_{off} described herein.

In some embodiments, there is provided an anti-B7-H3 humanized mAb or an antigen binding fragment thereof, comprising a V_{H} domain comprising the amino acid sequence of any one of SEQ ID NOs: 81-87, or a variant thereof having at least about 80% (such as at least about any of 80%, 85%, 90%, 91%, 92%, 93%, 94%, 95%, 96%, 97%, 98%, or 99%) sequence identify to any one of SEQ ID NOs: 81-87; and a VL domain comprising the amino acid sequence of any one of SEQ ID NOs: 88-91, or a variant thereof having at least about 80% (such as at least about any of 80%, 85%, 90%, 91%, 92%, 93%, 94%, 95%, 96%, 97%, 98%, or 99%) sequence identify to any one of SEQ ID NOs: 88-91. In some embodiments, there is provided an anti-B7-H3 mAb comprising a V_{H} domain comprising the amino acid sequence of any one of SEQ ID NOs: 81-87, or a variant thereof comprising up to about 3 (such as about any of 1, 2, or 3) amino acid substitutions in the V_{H} domain; and a VL domain comprising the amino acid sequence of any one of SEQ ID NOs: 88-91, or a variant thereof comprising up to about 3 (such as about any of 1, 2, or 3) amino acid substitutions in the VL domain. In some embodiments, an anti-B7-H3 mAb or an antigen binding fragment thereof comprises a variant of a V_{H} domain having the amino acid sequence of any one of SEQ ID NOs:81-87, wherein the variant comprises amino acid substitutions in CDRs, such as the CDR1, and/or the CDR2, and/or the CDR3 of the V_{H}; and a variant of a VL domain having the amino acid sequence of any one of SEQ ID NOs:88-91, wherein the variant comprises amino acid substitutions in CDRs, such as the CDR1, and/or the CDR2, and/or the CDR3 of any one of the VL. In some embodiments, an anti-B7-H3 mAb or an antigen binding fragment thereof comprises a variant of a V_{H} domain having the amino acid sequence of any one of SEQ ID NOs:81-87, wherein the variant comprises amino acid substitutions in FRs, such as the FR1, and/or the FR2, and/or the FR3, and/or the FR4 of any one of the V_{H}; and a variant of a VL domain having the amino acid sequence of any one of SEQ ID NOs: 88-91, wherein the variant comprises amino acid substitutions in FRs, such as the FR1, and/or the FR2, and/or the FR3, and/or the FR4.

In some embodiments, there is provided an anti-B7-H3 humanized mAb or an antigen binding fragment thereof, comprising: 1) a V_{H} comprising an amino acid sequence of SEQ ID NO: 81, and a VL comprising an amino acid sequence of SEQ ID NO: 88 (47F3F10B4-V_{H}1-VL4); 2) a V_{H} comprising an amino acid sequence of SEQ ID NO: 82, and a VL comprising an amino acid sequence of SEQ ID NO: 88 (47F3F10B4-V_{H}2-VL4); 3) a V_{H} comprising an amino acid sequence of SEQ ID NO: 83, and a VL comprising an amino acid sequence of SEQ ID NO: 89 (12C9F1B5-V_{H}2.1-VL2); 4) a V_{H} comprising an amino acid sequence of SEQ ID NO: 84, and a VL comprising an amino acid sequence of SEQ ID NO: 89 (12C9F1B5-V_{H}4-VL2); 5) a V_{H} comprising an amino acid sequence of SEQ ID NO: 85, and a VL comprising an amino acid sequence of SEQ ID NO: 90 (43H11G12-V_{H}4-VL2); 6) a V_{H} comprising an amino acid sequence of SEQ ID NO: 86, and a VL comprising an amino acid sequence of SEQ ID NO: 91 (31H11B1-V_{H}1-VL3); or 7) a V_{H} comprising an amino acid sequence of SEQ ID NO: 87, and a VL comprising an amino acid sequence of SEQ ID NO: 91 (31H11B1-V_{H}1.1-VL3).

In some embodiments, there is provided an anti-B7-H3 antibody, such as an mAb (hereinafter referred to as “competing anti-B7-H3 antibody or competing anti-B7-H3 mAb”), or an antigen binding fragment thereof, that specifically binds to B7-H3 competitively with any one of the anti-B7-H3 mAb described herein. In some embodiments, competitive binding can be determined using an ELISA assay. In some embodiments, the competing anti-B7-H3 antibody and the anti-B7-H3 antibody described above bind the same epitope on the B7-H3.

### Construct Comprising the Anti-B7-H3 mAb

The anti-B7-H3 construct comprising the anti-B7-H3 mAb can be of any possible format.

In some embodiments, the anti-B7-H3 construct comprising the anti-B7-H3 mAb can further comprise additional polypeptide sequences, such as one or more antibody moieties. Such additional polypeptide sequences can or cannot change or otherwise influence the (biological) properties of the anti-B7-H3 mAb, and can or cannot add further functionality to the anti-B7-H3 mAb described herein. In some embodiments, the additional polypeptide sequences confer one or more desired properties or functionalities to the anti-B7-H3 mAb of the application. In some embodiments, the anti-B7-H3 construct is a chimeric antigen receptor (CAR) comprising an extracellular antigen binding domain comprising one or more anti-B7-H3 binding moiety described herein.

In some embodiments, the additional polypeptide sequences can be a second antibody moiety (such as sdAb, scFv) that specifically recognizes a second antigen. In some embodiments, the second antigen is not B7-H3. In some embodiments, the second antibody moiety specifically recognizes the same epitope on B7-H3 as the anti-B7-H3 mAb described herein. In some embodiments, the second antibody moiety specifically recognizes a different epitope on B7-H3 as the anti-B7-H3 mAb described herein.

In some embodiments, the additional polypeptide sequences can increase the molecule's stability, solubility, or absorption, reduce immunogenicity or toxicity, eliminate or attenuate undesirable side effects, and/or confer other advantageous properties to and/or reduce undesired properties of the anti-B7-H3 construct of the invention, compared to the anti-B7-H3 mAb described herein per se.

### Full-Length IgG

In some embodiments, an anti-B7-H3 mAb is a full-length IgG. In some embodiments, the anti-B7-H3 mAb comprises the constant regions of IgG, such as any of IgG1, IgG2, IgG3, or IgG4. In some embodiments, the constant region is human constant region. In some embodiments, the constant region is human IgG1 constant region.

Thus in some embodiments, there is provided an anti-B7-H3 full-length IgG comprising a heavy chain, wherein the variable region (V_{H}) comprises a CDR1 comprising the amino acid sequence of any one of SEQ ID NOs: 2, 10, 18, 26, 34, 42, 50, 58, 66, and 74, or a variant thereof comprising up to about 3 (such as about any of 1, 2, or 3) amino acid substitutions; a CDR2 comprising the amino acid sequence of any one of SEQ ID NOs: 3, 11, 19, 27, 35, 43, 51, 59, 67, and 75, or a variant thereof comprising up to about 3 (such as about any of 1, 2, or 3) amino acid substitutions; and a CDR3 comprising the amino acid sequence of any one of SEQ ID NOs: 4, 12, 20, 28, 36, 44, 52, 60, 68, and 76, or a variant thereof comprising up to about 3 (such as about any of 1, 2, or 3) amino acid substitutions, and wherein the V_{H} is fused to the heavy chain constant regions (hinge, C_{H}1, C_{H}2 and C_{H}3) of an immunoglobulin; and a light chain, wherein the variable region (VL) comprises a CDR1 comprising the amino acid sequence of any one of SEQ ID NOs: 6, 14, 22, 30, 38, 46, 54, 62, 70, and 78, or a variant thereof comprising up to about 3 (such as about any of 1, 2, or 3) amino acid substitutions; a CDR2 comprising the amino acid sequence of any one of SEQ ID NOs: 7, 15, 23, 31, 39, 47, 55, 63, 71, and 79, or a variant thereof comprising up to about 3 (such as about any of 1, 2, or 3) amino acid substitutions; and a CDR3 comprising the amino acid sequence of any one of SEQ ID NOs: 8, 16, 24, 32, 40, 48, 56, 64, 72, and 80, or a variant thereof comprising up to about 3 (such as about any of 1, 2, or 3) amino acid substitutions, and wherein the VL is fused to the light chain constant region (CL) of an immunoglobulin. In some embodiments, there is provided an anti-B7-H3 full-length IgG comprising a heavy chain, wherein the variable region (V_{H}) comprises a CDR1 comprising the amino acid sequence of any one of SEQ ID NOs: 2, 10, 18, 26, 34, 42, 50, 58, 66, and 74; a CDR2 comprising the amino acid sequence of any one of SEQ ID NOs: 3, 11, 19, 27, 35, 43, 51, 59, 67, and 75; and a CDR3 comprising the amino acid sequence of any one of SEQ ID NOs: 4, 12, 20, 28, 36, 44, 52, 60, 68, and 76, or a variant thereof comprising up to about 3 (such as about any of 1, 2, or 3) amino acid substitutions, and wherein the V_{H} is fused to the heavy chain constant regions (hinge, C_{H}1, C_{H}2 and C_{H}3) of an immunoglobulin; and a light chain, wherein the variable region (VL) comprises a CDR1 comprising the amino acid sequence of any one of SEQ ID NOs: 6, 14, 22, 30, 38, 46, 54, 62, 70, and 78; a CDR2 comprising the amino acid sequence of any one of SEQ ID NOs: 7, 15, 23, 31, 39, 47, 55, 63, 71, and 79; and a CDR3 comprising the amino acid sequence of any one of SEQ ID NOs: 8, 16, 24, 32, 40, 48, 56, 64, 72, and 80, or a variant thereof comprising up to about 3 (such as about any of 1, 2, or 3) amino acid substitutions, and wherein the VL is fused to the light chain constant region (CL) of an immunoglobulin. In some embodiments, the full-length anti-B7-H3 mAb comprising a V_{H} comprising an amino acid sequence of any one of SEQ ID NOs: 1, 9, 17, 25, 33, 41, 49, 57, 65, and 73, and a VL comprising an amino acid sequence of any one of SEQ ID NOs: 5, 13, 21, 29, 37, 45, 53, 61, 69, and 77.

In some embodiments, the K_{D} of the binding between the full-length anti-B7-H3 IgG and B7-H3 is about 10⁻⁵ M to about 10⁻¹² M (such as about 10⁻⁷ M to about 10⁻¹² M, or about 10⁻⁸ M to about 10⁻¹² M), or less. In some embodiments, the full-length anti-B7-H3 IgG is rodent, chimeric, human, partially humanized, or fully humanized.

### Multivalent and/or Multispecific Antibodies

In some embodiments, the anti-B7-H3 construct comprises an anti-B7-H3 mAb described herein fused to one or more other antibody moiety (such as an antibody moiety that specifically recognizes another antigen). The one or more other antibody moiety can be of any antibody or antibody fragment format, such as an sdAb, a full-length antibody, a Fab, a Fab′, a (Fab′)₂, an Fv, a single chain Fv (scFv), an scFv-scFv, a minibody, or a diabody. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. For a review of multispecific antibodies, see Weidle et al., Cancer Genomics Proteomics, 10(1):1-18, 2013; Geering and Fussenegger, Trends Biotechnol., 33(2):65-79, 2015; Stamova et al., Antibodies, 1(2):172-198, 2012. Diabodies are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Nat'l. Acad. Sci. USA 90: 6444-6448 (1993). Triabodies and tetrabodies are also described in Hudson et al., Nat. Med. 9:129-134 (2003). Antibody fragments can be made by various techniques, including but not limited to proteolytic digestion of an intact antibody as well as production by recombinant host cells (e.g., E. coli or phage), as described herein. In some embodiments, the one or more other antibody moiety is antibody mimetics, which are small engineered proteins comprising antigen-binding domains reminiscent of antibodies (Geering and Fussenegger, Trends Biotechnol., 33(2):65-79, 2015). These molecules are derived from existing human scaffold proteins and comprise a single polypeptide. Exemplary antibody mimetics that can be comprised within the anti-B7-H3 construct described herein can be, but are not limited to, a designed ankyrin repeat protein (DARPin; comprising 3-5 fully synthetic ankyrin repeats flanked by N- and C-terminal Cap domains), an avidity multimer (avimer; a high-affinity protein comprising multiple A domains, each domain with low affinity for a target), or an Anticalin (based on the scaffold of lipocalins, with four accessible loops, the sequence of each can be randomized).

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies can also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Nat'l. Acad. Sci. USA, 90:6444-6448 (1993)); and using single-chain Fv (scFv) dimers (see, e.g., Gruber et al., J. Immunol., 152:5368 (1994)); and preparing trispecific antibodies as described, e.g., in Tutt et al. J. Immunol. 147: 60 (1991); and creating polypeptides comprising tandem single-domain antibodies (see, e.g., U.S. Patent Application No. 20110028695; and Conrath et al. J. Biol. Chem., 2001; 276(10):7346-50). Engineered antibodies with three or more functional antigen binding sites, including “Octopus antibodies,” are also included herein (see, e.g., US2006/0025576A1).

### Peptide Linkers

In some embodiments, the two or more antibody moieties within the anti-B7-H3 construct can be optionally connected by a peptide linker. The length, the degree of flexibility and/or other properties of the peptide linker(s) used in the anti-B7-H3 construct can have some influence on properties, including but not limited to the affinity, specificity or avidity for one or more particular antigens or epitopes. For example, longer peptide linkers can be selected to ensure that two adjacent domains do not sterically interfere with one another. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker can have a naturally occurring sequence, or a non-naturally occurring sequence. For example, a sequence derived from the hinge region of heavy chain only antibodies can be used as the linker. See, for example, WO1996/34103. In some embodiments, the peptide linker is a mutated human IgG1 hinge (EPKSSDKTHTSPPSP, SEQ ID NO: 95). In some embodiments, the peptide linker is a flexible linker. Exemplary flexible linkers include glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. In some embodiments, the peptide linker comprises the amino acid sequence of GGGGSGGGS (SEQ ID NO: 96). In some embodiments, the peptide linker comprises the amino acid sequence of SEQ ID NO: 97 (GGGGSGGGGSGGGGS).

### Bispecific Antibodies

In some embodiments, an isolated antibody or antigen binding fragment of the application is a bispecific or multispecific antibody that comprises an anti-B7-H3 IgG described herein fused to a second antibody moiety, wherein the second antibody moiety binds specifically to another antigen, preferably another inhibitory immune checkpoint molecules.

In an embodiment, the other antigen is CTLA-4 and the second antibody moiety comprises an antibody or antigen binding fragment that binds specifically to CTLA-4, such as an anti-CTLA-4 mAb, preferably an anti-CTLA-4 sdAb. The isolated antibody or antigen binding fragment comprising bi-specificity against B7-H3 and CTLA-4 can be hereinafter referred to as “anti-B7-H3/CTLA-4 antibody,” “anti-B7-H3/CTLA-4 construct,” or “B7-H3×CTLA-4 antibody.”

In an embodiment, the other antigen is PD-L1 and the second antibody moiety comprises an antibody or antigen binding fragment that binds specifically to PD-L1, such as an anti-PD-L1 mAb, preferably an anti-PD-L1 sdAb. The isolated antibody or antigen binding fragment comprising bi-specificity against B7-H3 and PD-L1 can be hereinafter referred to as “anti-B7-H3/PD-L1 antibody,” “anti-B7-H3/PD-L1 construct,” or “B7-H3×PD-L1 antibody.”

In an embodiment, the other antigen is TIM-3 and the second antibody moiety comprises an antibody or antigen binding fragment that binds specifically to TIM-3, such as an anti-TIM-3 mAb, preferably an anti-TIM3 sdAb. The isolated antibody or antigen binding fragment comprising bi-specificity against B7-H3 and TIM-3 can be hereinafter referred to as “anti-B7-H3/TIM-3 antibody,” “anti-B7-H3/TIM-3 construct,” or “B7-H3 xTIM-3 antibody.”

In an embodiment, the other antigen is LAG-3 and the second antibody moiety comprises an antibody or antigen binding fragment that binds specifically to LAG-3, such as an anti-LAG-3 mAb, preferably an anti-LAG-3 sdAb. The isolated antibody or antigen binding fragment having bi-specificity against B7-H3 and LAG-3 can be hereinafter referred to as “anti-B7-H3/LAG-3 antibody,” “anti-B7-H3/LAG-3 construct,” or “B7-H3×LAG-3 antibody.”

CTLA-4, PD-L1, TIM-3 and LAG-3 are inhibitory immune checkpoint molecules.

### Conjugates

Human monoclonal antibodies specific for B7-H3, or antigen binding fragments thereof, can be conjugated to an agent, such as an effector molecule or detectable marker, using any number of means known to those of skill in the art. Both covalent and non-covalent attachment means may be used. Conjugates include, but are not limited to, molecules in which there is a covalent linkage of an effector molecule or a detectable marker to an antibody or antigen binding fragment that specifically binds B7-H3. One of skill in the art will appreciate that various effector molecules and detectable markers can be used, including (but not limited to) chemotherapeutic agents, anti-angiogenic agents, toxins, radioactive agents such as ¹²⁵, ³²P, ¹⁴C, ³H and ³⁵S and other labels, target moieties and ligands, etc.

The choice of a particular effector molecule or detectable marker depends on the particular target molecule or cell, and the desired biological effect. Thus, for example, the effector molecule can be a cytotoxin that is used to bring about the death of a particular target cell (such as a tumor cell).

Effector molecules and detectable markers can be linked to an antibody or antigen binding fragment of interest using any number of means known to those of skill in the art. Both covalent and non-covalent attachment means may be used. The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, IL. The linker can be any molecule used to join the antibody or antigen binding fragment to the effector molecule or detectable marker. The linker is capable of forming covalent bonds to both the antibody or antigen binding fragment and to the effector molecule or detectable marker. Suitable linkers are well known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

Additionally, in several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference in its entirety.

Thus, in several embodiments, the conjugate includes a linker that connects the effector molecule or detectable marker to the B7-H3-specific antibody or antigen binding fragment thereof. In some embodiments, the linker is cleavable under intracellular conditions, such that cleavage of the linker releases the effector molecule or detectable marker from the antibody or antigen binding fragment in the intracellular environment. In yet other embodiments, the linker is not cleavable and the effector molecule or detectable marker is released, for example, by antibody degradation. In some embodiments, the linker is cleavable by a cleaving agent that is present in the intracellular environment (for example, within a lysosome or endosome or caveolea). The linker can be, for example, a peptide linker that is cleaved by an intracellular peptidase or protease enzyme, including, but not limited to, a lysosomal or endosomal protease. In some embodiments, the peptide linker is at least two amino acids long or at least three amino acids long. However, the linker can be 4, 5, 6, 7, 8, 9, 10, 11, 12, 13, 14 or 15 amino acids long, such as 1-2, 1-3, 2-5, 3-10, 3-15, 1-5, 1-10, 1-15, amino acids long. Proteases can include cathepsins B and D and plasmin, all of which are known to hydrolyze dipeptide drug derivatives resulting in the release of active drug inside target cells (see, for example, Dubowchik and Walker, 1999, Pharm. Therapeutics 83:67-123). For example, a peptide linker that is cleavable by the thiol-dependent protease cathepsin-B, can be used (for example, a Phenylalanine-Leucine or a Glycine-Phenylalanine-Leucine-Glycine linker). Other examples of such linkers are described, for example, in U.S. Pat. No. 6,214,345, incorporated herein by reference. In a specific embodiment, the peptide linker cleavable by an intracellular protease is a Valine-Citruline linker or a Phenylalanine-Lysine linker (see, for example, U.S. Pat. No. 6,214,345, which describes the synthesis of doxorubicin with the Valine-Citruline linker).

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) can be used. (See, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, for example, a thioether attached to the therapeutic agent via an acylhydrazone bond (see, for example, U.S. Pat. No. 5,622,929).

In yet other embodiments, the linker is cleavable under reducing conditions (for example, a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker is not cleavable and the effector molecule or detectable marker is released by antibody degradation. (See U.S. Publication No. 2005/0238649 incorporated by reference herein in its entirety).

In several embodiments, the linker is resistant to cleavage in an extracellular environment. For example, no more than about 20%, no more than about 15%, no more than about 10%, no more than about 5%, no more than about 3%, or no more than about 1% of the linkers, in a sample of conjugate, are cleaved when the conjugate is present in an extracellular environment (for example, in plasma). Whether or not a linker is resistant to cleavage in an extracellular environment can be determined, for example, by incubating the conjugate containing the linker of interest with plasma for a predetermined time period (for example, 2, 4, 8, 16, or 24 hours) and then quantitating the amount of free effector molecule or detectable marker present in the plasma. A variety of exemplary linkers that can be used in conjugates are described in WO2004-010957, U.S. Publication No. 2006/0074008, U.S. Publication No. 2005/0238649, and U.S. Publication No. 2006/0024317, each of which is incorporated by reference herein in its entirety.

The antibodies or antigen binding fragments disclosed herein can be derivatized, for example, by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

In view of the large number of methods that have been reported for attaching a variety of radiodiagnostic compounds, radiotherapeutic compounds, labels (such as enzymes or fluorescent molecules), toxins, and other agents to antibodies one skilled in the art will be able to determine a suitable method for attaching a given agent to an antibody or antigen binding fragment or other polypeptide. For example, the antibody or antigen binding fragment can be conjugated with small molecular weight drugs such as Monomethyl Auristatin E (MMAE), Monomethyl Auristatin F (MMAF), maytansine, maytansine derivatives, including the derivative of maytansine known as DM1 (also known as mertansine), or other chemotherapeutic agents to make an antibody drug conjugate (ADC). In several embodiments, various chemotherapeutic agents described herein can be conjugated to the provided antibodies to generate a conjugate.

In several embodiments, conjugates of an antibody or antigen binding fragment and one or more small molecule toxins, such as a calicheamicin, maytansinoids, dolastatins, auristatins, a trichothecene, and CC1065, and the derivatives of these toxins that have toxin activity, are provided.

Maytansine compounds suitable for use as maytansinoid toxin moieties are available and can be isolated from natural sources according to known methods, produced using genetic engineering techniques (see Yu et al (2002) PNAS 99:7968-7973), or maytansinol and maytansinol analogues prepared synthetically according to known methods. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, each of which is incorporated herein by reference. Conjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference.

### Anti-B7-H3 Antibody Variants

In some embodiments, amino acid sequence variants of the antibodies provided herein are contemplated. For example, it can be desirable to improve the binding affinity and/or other biological properties of the antibody. Amino acid sequence variants of an antibody can be prepared by introducing appropriate modifications into the nucleic acid sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, Deletion and Variants

In some embodiments, antibody variants having one or more amino acid substitutions are provided. Sites of interest for substitutional mutagenesis include the HVRs and FRs. Conservative substitutions are shown in Table 3 under the heading of “Preferred substitutions.” More substantial changes are provided in Table 3 under the heading of “exemplary substitutions,” and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 3
Amino acid substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids can be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) can be made in HVRs, e.g., to improve antibody affinity. Such alterations can be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding can be specifically identified, e.g., using alanine scanning mutagenesis or modeling. CDR-H3 and CDR-L3 in particular are often targeted.

In some embodiments, substitutions, insertions, or deletions can occur within one or more HVRs so long as such alterations do not substantially reduce the ability of the antibody to bind antigen. For example, conservative alterations (e.g., conservative substitutions as provided herein) that do not substantially reduce binding affinity can be made in HVRs. Such alterations can be outside of HVR “hotspots” or CDRs. In some embodiments of the variant V_{H}H sequences provided above, each HVR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In some embodiments, an anti-B7-H3 construct provided herein is altered to increase or decrease the extent to which the construct is glycosylated. Addition or deletion of glycosylation sites to an antibody can be conveniently accomplished by altering the amino acid sequence such that one or more glycosylation sites is created or removed.

Where the anti-B7-H3 construct comprises an Fc region, the carbohydrate attached thereto can be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-B7-H3 construct of the present application can be made in order to create antibody variants with certain improved properties.

In some embodiments, antibody variants are provided having a carbohydrate structure that lacks fucose attached (directly or indirectly) to an Fc region. For example, the amount of fucose in such antibody can be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 can also be located about ±3 amino acids upstream or downstream of position 297, i.e., between positions 294 and 300, due to minor sequence variations in antibodies. Such fucosylation variants can have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-B7-H3 construct variants are further provided with bisected oligosaccharides, e.g., in which a biantennary oligosaccharide attached to the Fc region of the antibody is bisected by GlcNAc. Such antibody variants can have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants can have improved CDC function. Such antibody variants are described, e.g., in WO 1997/30087 (Patel et al.); WO 1998/58964 (Raju, S.); and WO 1999/22764 (Raju, S.).

### c) Fc Region Variants

In some embodiments, one or more amino acid modifications can be introduced into the Fc region of the anti-B7-H3 construct provided herein, thereby generating an Fc region variant. The Fc region variant can comprise a human Fc region sequence (e.g., a human IgG1, IgG2, IgG3 or IgG4 Fc region) comprising an amino acid modification (e.g., a substitution) at one or more amino acid positions.

In some embodiments, the present application contemplates an anti-B7-H3 construct variant that possesses some but not all effector functions, which make it a desirable candidate for applications in which the half-life of the anti-B7-H3 construct in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (Cell Technology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an anti-B7-H3 construct variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, there is provided an anti-B7-H3 construct (e.g., a HCAb) variant comprising a variant Fc region comprising one or more amino acid substitutions which increase half-life and/or improve binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fe region with one or more substitutions therein which improve binding of the Fe region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-B7-H3 constructs (such as full-length IgG or anti-B7-H3 IgG fused to an sdAb) comprising any of the Fc variants described herein, or combinations thereof, are contemplated.

In some embodiments, the constant regions are human IgG1 constant region. In some embodiments, the heavy chain constant region comprises a modification that enhances the ADCC effect. In some embodiments, the heavy chain constant region comprises mutations K214R, L235V, F243L, R292P, Y300L, D356E, L358M and P396L. In some embodiments, the heavy chain constant region comprises an amino acid sequence of SEQ ID NO: 93 or 94. In some embodiments, the light chain constant region comprises an amino acid sequence of SEQ ID NO: 92.

### d) Cysteine Engineered Antibody Variants

In some embodiments, it can be desirable to create cysteine engineered anti-B7-H3 constructs, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. In some embodiments, any one or more of the following residues can be substituted with cysteine: A118 (EU numbering) of the heavy chain; and S400 (EU numbering) of the heavy chain Fc region. Cysteine engineered anti-B7-H3 constructs can be generated as described, e.g., in U.S. Pat. No. 7,521,541.

### e) Antibody Derivatives

In some embodiments, an anti-B7-H3 construct provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (eitherhomopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of an anti-B7-H3 construct and nonproteinaceous moiety that can be selectively heated by exposure to radiation are provided. In some embodiments, the nonproteinaceous moiety is a carbon nanotube (Kam et al., Proc. Nat'l. Acad. Sci. USA 102: 11600-11605 (2005)). The radiation can be of any wavelength, and includes, but is not limited to, wavelengths that do not harm ordinary cells, but which heat the nonproteinaceous moiety to a temperature at which cells proximal to the antibody-nonproteinaceous moiety are killed.

In some embodiments, an anti-B7-H3 construct provided herein (such as anti-B7-H3 IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody) can be further modified to contain one or more biologically active protein, polypeptides or fragments thereof. “Bioactive” or “biologically active” as used herein means showing biological activity in the body to carry out a specific function. For example, it can mean the combination with a particular biomolecule such as protein, DNA, etc., and then promotion or inhibition of the activity of such biomolecule. In some embodiments, the bioactive protein or fragments thereof have immunostimulatory/immunoregulatory, membrane transport, or enzymatic activities.

In some embodiments, the bioactive protein or fragments thereof that can be fused with the anti-B7-H3 construct described herein is a ligand, such as lymphokines and cellular factors which interact with specific cellular receptor. Lymphokines are low molecular weight proteins which are secreted by T cells when antigens or lectins stimulate T cell growth. Examples of lymphokines include, but are not limited to, interferon-α, interferon-γ, interleukin-1 (IL-1), interleukin-2 (IL-2), interleukin-3 (IL-3), tumor necrosis factor (TNF), a colony stimulating factor (e.g. CSF-1, G-CSF or GM-CSF), chemotaxins, macrophage migration inhibitory factor (MIF), macrophage-activating factor (MAF), NK cell activating factor, T cell replacing factor, leukocyte-inhibitory factor (LIF), lymphotoxins, osteoclast-activating factor (OAF), soluble immune response suppressor (SIRS), growth-stimulating factor, monocyte growth factor, etc. Cellular factors which can be incorporated into the anti-B7-H3 fusion proteins of the invention include but are not limited to tumor necrosis factor α (TNFα), interferons (IFNs), and nerve growth factor (NGF), etc.

### III. Pharmaceutical Compositions

Further provided by the present application are pharmaceutical compositions comprising any one of the anti-B7-H3 constructs (such as anti-B7-H3 IgG, full-length anti-B7-H3 IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody), and optionally a pharmaceutically acceptable carrier. Pharmaceutical compositions can be prepared by mixing an anti-B7-H3 construct described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

The pharmaceutical composition is preferably to be stable, in which the anti-B7-H3 construct comprising anti-B7-H3 mAb described here essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation can be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of anti-B7-H3 construct described herein can comprise less than about 10% (preferably less than about 5%) of the anti-B7-H3 construct present as an aggregate in the formulation.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g., sodium chloride), stabilizers, metal complexes (e.g., Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™ polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers can comprise histidine and trimethylamine salts such as Tris.

Preservatives are added to retard microbial growth, and are typically present in a range from 0.2%-1.0% (w/v). The addition of a preservative can, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition can be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally, such as intratumorally.

Sustained-release preparations can be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and. Ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein can also contain more than one active compound as necessary for the particular indication being treated, preferably those with complementary activities that do not adversely affect each other. Alternatively, or in addition, the composition can comprise a cytotoxic agent, chemotherapeutic agent, cytokine, immunosuppressive agent, or growth inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18^{th} edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### IV. Methods of Uses or Applications

The anti-B7-H3 construct comprising mAb specifically recognizing B7-H3 as described herein (such as anti-B7-H3 full-length IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody), and the compositions (such as pharmaceutical compositions) thereof are useful for a variety of applications, such as in diagnosis, molecular assays, and therapy.

One aspect of the invention provides a method of treating a B7-H3 related disease or a condition in an individual in need thereof, comprising administering to the individual an effective amount of a pharmaceutical composition comprising the anti-B7-H3 construct described herein. In some embodiments, the B7-H3 related disease is cancer. In some embodiments, the cancer is a non-small cell lung cancer (NSCLC), a head and neck squamous cell carcinoma (HNSCC), or a breast cancer.

The application contemplates, in part, protein constructs (such as anti-B7-H3 full-length IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody), nucleic acid molecules and/or vectors encoding thereof, host cells comprising nucleic acid molecules and/or vectors encoding thereof, that can be administered either alone or in any combination with another therapy, and in at least some aspects, together with a pharmaceutically acceptable carrier or excipient. In some embodiments, prior to administration of the anti-B7-H3 construct, they can be combined with suitable pharmaceutical carriers and excipients that are well known in the art. The compositions prepared according to the disclosure can be used for the treatment or delaying of worsening of cancer.

In some embodiments, there is provided a method of treating cancer comprising administering to the individual an effective amount of a pharmaceutical composition comprising an isolated anti-B7-H3 construct comprising a mAb specifically recognizing B7-H3 (such as anti-B7-H3 full-length IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody). In some embodiments, the cancer is a solid tumor (such as lung cancer). In some embodiments, the pharmaceutical composition is administered systemically (such as intravenously). In some embodiments, the pharmaceutical composition is administered locally (such as intratumorally). In some embodiments, the method further comprises administering to the individual an additional cancer therapy (such as surgery, radiation, chemotherapy, immunotherapy, hormone therapy, or a combination thereof). In some embodiments, the individual is a human. In some embodiments, the method of treating cancer has one or more of the following biological activities: (1) killing cancer cells (including bystander killing); (2) inhibiting proliferation of cancer cells; (3) inducing immune response in a tumor; (4) reducing tumor size; (5) alleviating one or more symptoms in an individual having cancer; (6) inhibiting tumor metastasis; (7) prolonging survival; (8) prolonging time to cancer progression; and (9) preventing, inhibiting, or reducing the likelihood of the recurrence of a cancer. In some embodiments, the method of killing cancer cells mediated by the pharmaceutical composition described herein can achieve a tumor cell death rate of at least about any of 40%, 50%, 60%, 70%, 80%, 90%, 95%, or more. In some embodiments, the method of killing cancer cells mediated by the pharmaceutical composition described herein can achieve a bystander tumor cell (uninfected by the oncolytic VV) death rate of at least about any of 10%, 20%, 30%, 40%, 50%, 60%, 70%, 80%, 90%, 95%, or more. In some embodiments, the method of reducing tumor size mediated by the pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the pharmaceutical composition described herein can inhibit at least about 10% (including for example at least about any of 20%, 30%, 40%, 60%, 70%, 80%, 90%, or 100%) of the metastasis. In some embodiments, the method of prolonging survival of an individual (such as a human) mediated by the pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the pharmaceutical composition described herein can prolongs the time to cancer progression by at least any of 1, 2, 3, 4, 5, 6, 7, 8, 9, 10, 11, or 12 weeks.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein can be used as a first therapy, second therapy, third therapy, or combination therapy with other types of cancer therapies known in the art, such as chemotherapy, surgery, hormone therapy, radiation, gene therapy, immunotherapy (such as T-cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method can be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

In some embodiments, the method is suitable for treating cancers with aberrant B7-H3 expression, activity and/or signaling include, by way of non-limiting example, a bladder cancer, a cervical cancer, a colon cancer, a colorectal cancer, a gastric cancer, a liver cancer, a lung cancer, an ovarian cancer, a pancreatic cancer, a prostate cancer, a kidney cancer, a breast cancer, a head and neck cancer, a skin cancer, a sarcoma, a brain tumor, a brain and spinal cord cancer, an adrenal cancer, a uterine cancer, a neuroblastoma, a small round cell tumor, a peripheral nerve sheath tumor, a bone cancer, a rhabdoid tumor, a lymphoma, a multiple myeloma, a leukemia, a neuroendocrine tumor, and a melanoma.

Thus in some embodiments, there is provided a method of treating an immunotherapy-responsive solid tumor (such as carcinoma or adenocarcinoma, such as cancers with aberrant B7-H3 expression, activity and/or signaling), comprising administering to the individual an effective amount of a pharmaceutical composition comprising an isolated anti-B7-H3 construct comprising a monoclonal antibody specifically recognizing B7-H3 (such as anti-B7-H3 full-length IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody). In some embodiments, the cancer is a solid tumor (such as lung cancer). In some embodiments, the pharmaceutical composition is administered systemically (such as intravenously). In some embodiments, the pharmaceutical composition is administered locally (such as intratumorally). In some embodiments, the method further comprises administering to the individual an additional cancer therapy (such as surgery, radiation, chemotherapy, immunotherapy, hormone therapy, or a combination thereof). In some embodiments, the individual is a human. In some embodiments, the method of treating cancer has one or more of the following biological activities: (1) killing cancer cells (including bystander killing); (2) inhibiting proliferation of cancer cells; (3) inducing immune response in a tumor; (4) reducing tumor size; (5) alleviating one or more symptoms in an individual having cancer; (6) inhibiting tumor metastasis; (7) prolonging survival; (8) prolonging time to cancer progression; and (9) preventing, inhibiting, or reducing the likelihood of the recurrence of a cancer. In some embodiments, the method of killing cancer cells mediated by the pharmaceutical composition described herein can achieve a tumor cell death rate of at least about any of 40%, 50%, 60%, 70%, 80%, 90%, 95%, or more. In some embodiments, the method of killing cancer cells mediated by the pharmaceutical composition described herein can achieve a bystander tumor cell (uninfected by the oncolytic VV) death rate of at least about any of 10%, 20%, 30%, 40%, 50%, 60%, 70%, 80%, 90%, 95%, or more. In some embodiments, the method of reducing tumor size mediated by the pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the pharmaceutical composition described herein can inhibit at least about 10% (including for example at least about any of 20%, 30%, 40%, 60%, 70%, 80%, 90%, or 100%) of the metastasis. In some embodiments, the method of prolonging survival of an individual (such as a human) mediated by the pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the pharmaceutical composition described herein can prolongs the time to cancer progression by at least any of 1, 2, 3, 4, 5, 6, 7, 8, 9, 10, 11, or 12 weeks.

Dosages and desired drug concentrations of pharmaceutical compositions of the present application can vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the anti-B7-H3 construct comprising an anti-B7-H3 mAb described herein are used, normal dosage amounts can vary from about 10 ng/kg up to about 100 mg/kg of mammal body weight or more per day, preferably about 1 mg/kg/day to 10 mg/kg/day, such as about 1-3 mg/kg/day, about 2-4 mg/kg/day, about 3-5 mg/kg/day, about 4-6 mg/kg/day, about 5-7 mg/kg/day, about 6-8 mg/kg/day, about 6-6.5 mg/kg/day, about 6.5-7 mg/kg/day, about 7-9 mg/kg/day, or about 8-10 mg/kg/day, depending upon the route of administration. It is within the scope of the present application that different formulations will be effective for different treatments and different disorders, and that administration intended to treat a specific organ or tissue can necessitate delivery in a manner different from that to another organ or tissue. Moreover, dosages can be administered by one or more separate administrations, or by continuous infusion. For repeated administrations over several days or longer, depending on the condition, the treatment is sustained until a desired suppression of disease symptoms occurs. However, other dosage regimens can be useful. The progress of this therapy is easily monitored by conventional techniques and assays.

In some embodiments, the pharmaceutical composition is administered for a single time (e.g. bolus injection). In some embodiments, the pharmaceutical composition is administered for multiple times (such as any of 2, 3, 4, 5, 6, or more times). If multiple administrations, they can be performed by the same or different routes and can take place at the same site or at alternative sites. The pharmaceutical composition can be administered twice per week, 3 times per week, 4 times per week, 5 times per week, daily, daily without break, once per week, weekly without break, once per 2 weeks, once per 3 weeks, once per month, once per 2 months, once per 3 months, once per 4 months, once per 5 months, once per 6 months, once per 7 months, once per 8 months, once per 9 months, once per 10 months, once per 11 months, or once per year. The interval between administrations can be about any one of 24 h to 48 h, 2 days to 3 days, 3 days to 5 days, 5 days to 1 week, 1 week to 2 weeks, 2 weeks to 1 month, 1 month to 2 months, 2 months to 3 months, 3 months to 6 months, or 6 months to a year. Intervals can also be irregular (e.g., following tumor progression). In some embodiments, there is no break in the dosing schedule. In some embodiments, the pharmaceutical composition is administered every 4 days for 4 times. The optimal dosage and treatment regime for a particular patient can readily be determined by one skilled in the art of medicine by monitoring the patient for signs of disease and adjusting the treatment accordingly.

The pharmaceutical compositions of the present application, including but not limited to reconstituted and liquid formulations, are administered to an individual in need of treatment with the anti-B7-H3 construct described herein, preferably a human, in accord with known methods, such as intravenous administration as a bolus or by continuous infusion over a period of time, by intramuscular, intraperitoneal, intracerobrospinal, subcutaneous, intravenous (i.v.), intra-articular, intrasynovial, intrathecal, oral, topical, or inhalation routes. A reconstituted formulation can be prepared by dissolving a lyophilized anti-B7-H3 construct described herein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical compositions are administered to the individual by subcutaneous (i.e., beneath the skin) administration. For such purposes, the pharmaceutical compositions can be injected using a syringe. However, other devices for administration of the pharmaceutical compositions are available such as injection devices; injector pens; auto-injector devices, needleless devices; and subcutaneous patch delivery systems.

In some embodiments, the pharmaceutical compositions are administered to the individual intravenously. In some embodiments, the pharmaceutical composition is administered to an individual by infusion, such as intravenous infusion. Infusion techniques for immunotherapy are known in the art (see, e.g., Rosenberg et al., New Eng. J. of Med. 319: 1676 (1988)).

The anti-B7-H3 construct comprising mAb specifically recognizing B7-H3 as described herein (such as anti-B7-H3 full-length IgG, anti-B7-H3/CTLA-4 bispecific antibody, anti-B7-H3/PD-L1 bispecific antibody, anti-B7-H3/TIM-3 bispecific antibody or anti-B7-H3/LAG-3 bispecific antibody), and the compositions (such as pharmaceutical compositions) thereof are also useful in diagnosis or molecular assays. For example, the antibody or antigen binding fragment can be used for the detection or quantification of B7-H3 in a biological sample, thereby detecting or monitoring the progress or treatment of a disease, such as those described above, related to B7-H3.

Administration of the antibodies, antigen binding fragments, conjugates, bispecific antibodies or compositions can be accompanied by administration of other anti-cancer or anti-angiogenesis agents or therapeutic treatments (such as surgical resection of a tumor or radiation therapy). For example, prior to, during, or following administration of a therapeutic amount of the antibodies or conjugates, the subject can receive one or more additional therapies. In one example, the subject receives one or more treatments to remove or reduce the tumor prior to administration of a therapeutic amount of one or more agents for treatment of the tumor. For example, the additional agent may include, but is not limited to, a chemotherapeutic agent, an anti-angiogenic agent, or a combination thereof. In another example, at least part of the tumor is surgically or otherwise excised or reduced in size or volume prior to administering the therapeutically effective amount of the antibody or antigen binding fragment or conjugate.

Particular examples of additional therapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. These agents (which are administered at a therapeutically effective amount) and treatments can be used alone or in combination. For example, any suitable anti-cancer or anti-angiogenic agent can be administered in combination with the antibodies, conjugates disclosed herein. Methods and therapeutic dosages of such agents are known to those skilled in the art, and can be determined by a skilled clinician. In one example the chemotherapeutic agent includes 5-FU or IRT or both.

### V. Methods of Preparation

The anti-B7-H3 construct (such as anti-B7-H3 monoclonal antibody) described herein can be prepared using any methods known in the art or as described herein.

Rodent monoclonal antibodies can be obtained using methods known in the art such as by immunizing a rodent species (such as mouse or rat) and obtaining hybridomas therefrom, or by cloning a library of Fab fragment or single chain Fc (scFv) using molecular biology techniques known in the art and subsequent selection by ELISA with individual clones of unselected libraries or by using phage display.

For recombinant production of the monoclonal antibodies, the nucleic acids encoding the monoclonal antibodies are isolated or synthesized and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

### EXAMPLES

The examples below are intended to be purely exemplary of the invention and should therefore not be considered to limit the invention in any way. The following examples and detailed description are offered by way of illustration and not by way of limitation.

### Example 1: Generation of Anti-B7-H3 Monoclonal Antibodies (mAbs)

### Immunization

A/J mice were immunized with 25 μg of human B7-H3 protein (AcroBiosystems, cat. no. B7B-H52E7) formulated as an emulsion with adjuvants (CFA, Complete Freund's Adjuvant, for primary immunization; IFA, Incomplete Freund's Adjuvant, for boost immunizations) in a volume of 200 μl subcutaneously under abdominal skin. Each mouse received 3-5 doses. Seven days after each time point of immunization (except the last immunization), 20 μl of blood sample were collected from the animals to monitor the anti-sera titer in an ELISA assay with immobilized human B7-H3-His proteins till the fusion criteria was met.

### Selection of Anti-Human B7-H3 Antibody Secreting Hybridoma

Three days after the last immunization, splenocytes from selected mice were extracted and fused with SP2/0 cells following standard hybridoma generation protocol in sterile environment. The fused cells were cultured in 1× HAT (hypoxanthin-aminopterin-thymidine) containing DMEM media, supplemented with 10% FBS for seven days. The hybridoma supernatants were analyzed for their binding ability to human B7-H3, cyno B7-H3 or mouse B7-H3 respectively by ELISA. The positive hybridoma clones were sub-cloned by limited dilution and cultured in 1× HT (hypoxanthine-thymidine) containing DMEM media, supplemented with 10% FBS. The cells were cultured for one week before a new round of screening till positive single clones were obtained. Each clone was used to produce 0.5 mg antibodies which were purified using protein A chromatography for further characterization. Antibody isotypes were tested using Clonotyping system-HRP, Southern Biotech.

### Example 2: Characterization of B7-H3 Mouse Antibodies In Vitro

### CHO-K1/B7-H3 Cell Binding by FACS

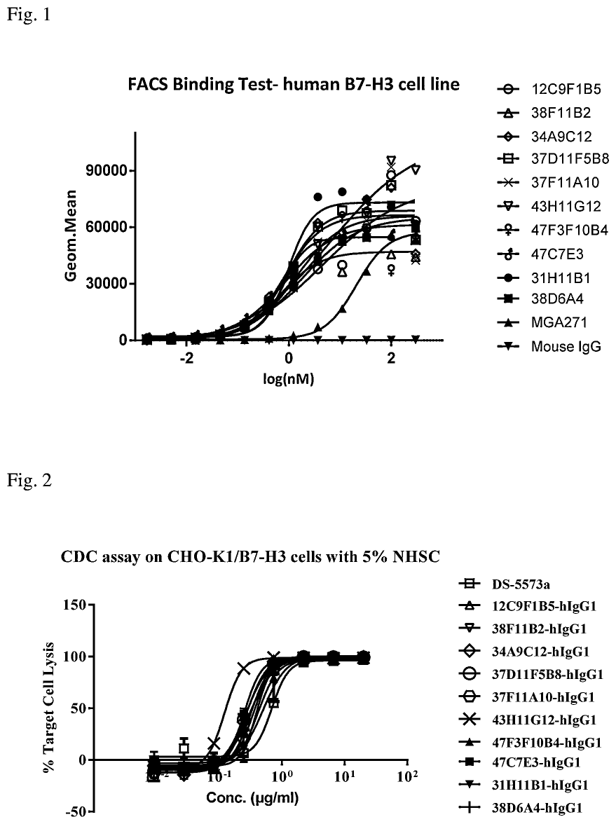

Purified antibodies from positive clones were used for FACS binding assays against CHO-K1/B7-H3 cell line (Probio Biotech), where binding EC₅₀ values were generated (FIG. 1).

Positive clones include 12C9F1B5, 38F11B2, 34A9C12, 37D11F5B8, 37F11A10, 43H11G12, 47F3F10B4, 47C7E3, 31H11B1, 38D6A4, MGA271. Mouse IgG was used as the isotype control.

EC₅₀ values for the FACS binding assay are given in Table 4. All the positive clones have higher binding affinity than the reference antibody of MGA271 (Enoblituzumab).

TABLE 4
Mouse mAb FACS binding analysis

| Clone No. | FACS binding EC₅₀ value (nM) |
|---|---|
| 12C9F1B5 | 4.759 |
| 38F11B2 | 0.6511 |
| 34A9C12 | 0.8217 |
| 37D11F5B8 | 0.9091 |
| 37F11A10 | 1.377 |
| 43H11G12 | 6.375 |
| 47F3F10B4 | 0.7509 |
| 47C7E3 | 0.8696 |
| 31H11B1 | 0.9766 |
| 38D6A4 | 1.777 |
| MGA271 | 20.82 |
| Mouse IgG | Not Applicable |

### Example 3: Chimeric Antibody Production and Analysis

### Construction of Chimeric Antibodies for Characterization

Variable region coding sequences for these 10 mAbs were optimized for human codon biased expression with GenScript online tools. The variable DNA fragments were synthesized and fused to human IgG1 heavy chain domains (C_{H}1-hinge-C_{H}2-C_{H}3) and light chain kappa constant regions (CL) for transient expression in chimeric formats. The heavy chain and light chain expression constructs were cloned into individual pTT5 based plasmids after synthesized signal peptide for secretory expression.

The chimeric antibodies were expressed in HEK293-6E cells transfected with antibody heavy chain/light pair plasmids using PEImax 40,000 (polysciences). 24 hours later, the expression/secretion was boosted with Tryptone N-1 supplement (Kirgen Bioscience). After 5 days of shaking culture in 37° C., 5% CO₂, supernatants were collected and the antibody content was purified with Protein-A beads (Genscript). Chimeric antibody products were kept in PBS for analysis.

### Anti-B7-H3 Antibody Induced CDC Analysis

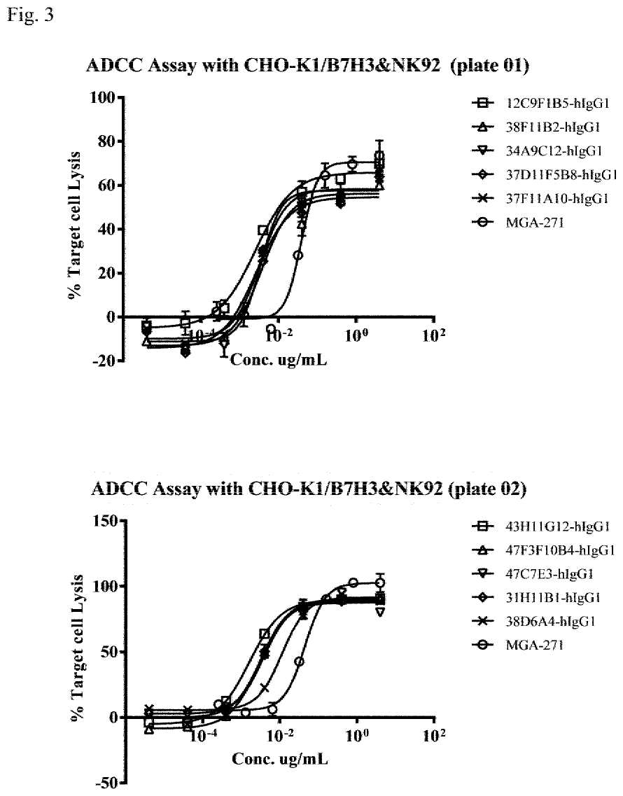

CDC assay was conducted for the chimeric antibodies. The target cell line, CHO-K1-overexpressing human B7-H3, was cultured and harvested, and it was seeded in 384-well plate at a specific cell density (5E3 cells/well). Antibody samples were added to the plate accordingly and the plate was incubated at 37° C./5% CO₂ for 30 min. Purified normal human serum complement (NHSC, SIGMA-ALDRICH) was then added to the plate and the plate was incubated further for 4 hours. The plate was taken out of the incubator and cells viability analyzed with Cell Titer-Glo® assay kit (Promega). The luminescence data was captured by PheraStar (BMG) for cell viability analysis. The CDC assay results in term of % target cell lysis versus candidate antibody concentration are shown in FIG. 2.

The EC₅₀ values of all the candidate chimeric antibodies from CDC assay are summarized in table 5. Clone 43H11G12-hIgG1 shows the lowest EC₅₀ value of 0.1217 μg/ml. The EC₅₀ value of the in-house synthesized positive control which has the identical amino acid sequence as DS-5573a (Patent NO: US 2013/0078234 A1) was used to calculate the relative activities for each candidate antibody (% Relative activity=(EC₅₀ of the positive control/EC₅₀ of the sample)*100%). All chimeric mAb samples showed more than 100% relative activity.

TABLE 5
Chimeric antibody CDC induction analysis

| Sample ID | EC₅₀(μg/ml) | % Relative activity |
|---|---|---|
| 12C9F1B5-hIgG1 | 0.2995 | 230.32 |
| 38F11B2-hIgG1 | 0.3187 | 216.44 |
| 34A9C12-hIgG1 | 0.2686 | 256.81 |
| 37D11F5B8-hIgG1 | 0.2673 | 258.06 |
| 37F11A10-hIgG1 | 0.3574 | 193.01 |
| 43H11G12-hIgG1 | 0.1217 | 566.80 |
| 47F3F10B4-hIgG1 | 0.4178 | 165.10 |
| 47C7E3-hIgG1 | 0.251 | 274.82 |
| 31H11B1-hIgG1 | 0.3925 | 175.75 |
| 38D6A4-hIgG1 | 0.5034 | 137.03 |
| DS-5573a | 0.6898 | 100.00 |

### Anti-B7-H3 Antibody Induced ADCC Assay

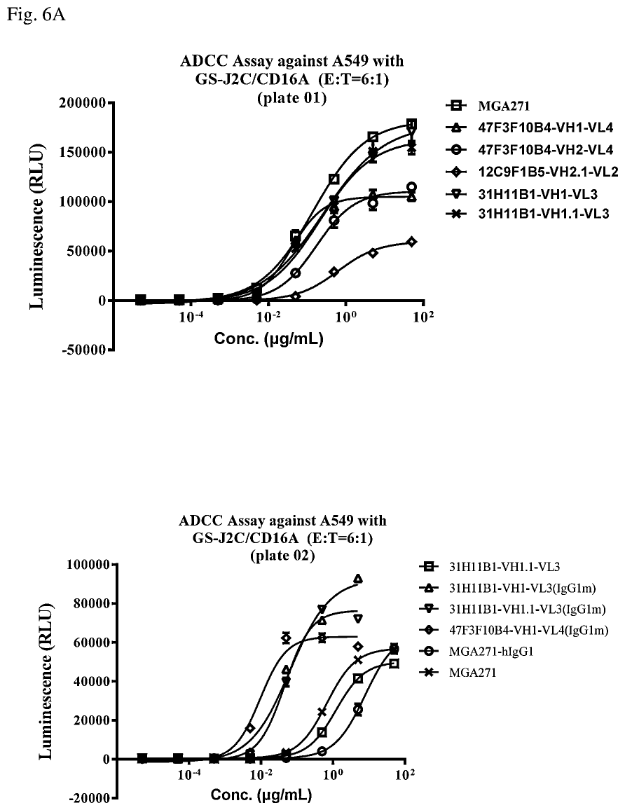

For the assay procedure, the target cell line, CHO-K1-overexpressing human B7-H3 (Probio Biotech), was cultured, harvested and seeded into 96 well plates at a specific cell density. The chimeric antibody samples or positive control, which was in-house synthesized with the identical amino acid sequence of MGA-271 (Enoblituzumab) (Patent NO: US 2012/0294796 A1), were added to the plates and the plates were incubated at 37° C./5% CO₂ for 30 min. NK92/CD16a-VV cells were used as the effector cells and added to the plates and incubated at the same condition for 6 hours. The assay plates were taken out and short centrifuged. The supernatant was collected and transferred to a new plate for LDH activity assay as per manufacturer's instruction (Roche). The absorbance data were captured by PheraStar (BMG) and analyzed by GraphPad Prism 6.0. The ADCC effect of all the chimeric antibodies were compared. The ADCC assay results in term of % target cell lysis versus candidate antibody concentration are shown in FIG. 5.

The EC₅₀ values of the following candidate chimeric antibodies from ADCC assay (two plates in one experiment) are summarized in table 6. Clone 43H11G12-hIgG1 shows the lowest EC₅₀ value of 0.001615 μg/ml. The EC₅₀ value of the in-house synthesized positive control which has the identical amino acid sequence as MGA-271 (Enoblituzumab) (Patent NO: US 2012/0294796 A1) was used to calculate the relative activities for each candidate antibody on every assay plate (% Relative activity=(EC₅₀ of the positive control/EC₅₀ of the sample)*100). All chimeric mAb samples showed more than 300% relative activity.

TABLE 6
Chimeric antibody ADCC induction analysis

| Plate No. | Sample ID | EC₅₀(μg/ml) | % Relative activity |
|---|---|---|---|
| plate 01 | 12C9F1B5-hIgG1 | 0.002585 | 1524.56 |
| | 38F11B2-hIgG1 | 0.002656 | 1483.81 |
| | 34A9C12-hIgG1 | 0.003411 | 1155.38 |
| | 37D11F5B8-hIgG1 | 0.00368 | 1070.92 |
| | 37F11A10-hIgG1 | 0.003339 | 1180.29 |
| | MGA-271 | 0.03941 | 100.00 |
| plate 02 | 43H11G12-hIgG1 | 0.001615 | 2744.89 |
| | 47F3F10B4-hIgG1 | 0.002892 | 1532.85 |
| | 47C7E3-hIgG1 | 0.003684 | 1203.31 |
| | 31H11B1-hIgG1 | 0.003254 | 1362.32 |
| | 38D6A4-hIgG1 | 0.01132 | 391.61 |
| | MGA-271 | 0.04433 | 100.00 |

### Example 4. Humanization Antibody Production and Analysis

### Humanization Design for the Candidate Antibodies

Based on antibodies' potency in function assays and their variable domain sequences, the CDRs and FRs were analyzed and homology modeling was performed to obtain the modeled structure of the mAbs of 47F3F10B4, 12C9F1B5, 43H11G12 and 31H11B1. The solvent accessible surface area of framework residues was calculated. Based on the result, framework residues that are buried (i.e. with solvent accessible surface area of <15%) were identified. The human acceptors for V_{H} and VL that share the high sequences identity to the mouse counterparts were selected. The CDRs of the mouse antibody were directly grafted to the human acceptor frameworks to obtain the sequence of the grafted antibody without any back mutation. Post translational modifications and chemical degradation in grafted sequence including deamidation, isomerization oxidation and glycosylation, etc., were analyzed through developability assessment. PTM hotspots like N-glycosylation sites, unusual proline residues, deamidation site, isomerization site, oxidation site and unpaired cysteine residues etc. that may affect the binding activity and manufacturability of the grafted antibody, were identified. The DNA sequences encoding the humanized light and heavy chains were synthesized. The antibody characteristics were compared to select the best candidate. These humanized molecules were successfully produced.

### Humanized Antibody Production

The humanized heavy chain and light chain of antibodies were combined for antibody production. For each heavy chain and light chain combination, the detailed information is shown in Table 7. Meanwhile, the heavy chain mutant of human IgG1 (K214R, L235V, F243L, R292P, Y300L, D356E, L358M and P396L) called IgG1m was utilized to enhance ADCC effect.

TABLE 7
Humanized antibody production by heavy
chain and light chain pairing

| Antibody name | Heavy chain | Light chain |
|---|---|---|
| 47F3F10B4-VH1-VL4 | 47F3F10B4-VH1 | 47F3F10B4-VL4 |
| 47F3F10B4-VH2-VL4 | 47F3F10B4-VH2 | 47F3F10B4-VL4 |
| 12C9F1B5-VH2.1-VL2 | 12C9F1B5-VH2.1 | 12C9F1B5-VL2 |
| 12C9F1B5-VH4-VL2 | 12C9F1B5-VH4 | 12C9F1B5-VL2 |
| 43H11G12-VH4-VL2 | 43H11G12-VH4 | 43H11G12-VL2 |
| 31H11B1-VH1-VL3 | 31H11B1-VH1 | 31H11B1-VL3 |
| 31H11B1-VH1.1-VL3 | 31H11B1-VH1.1 | 31H11B1-VL3 |

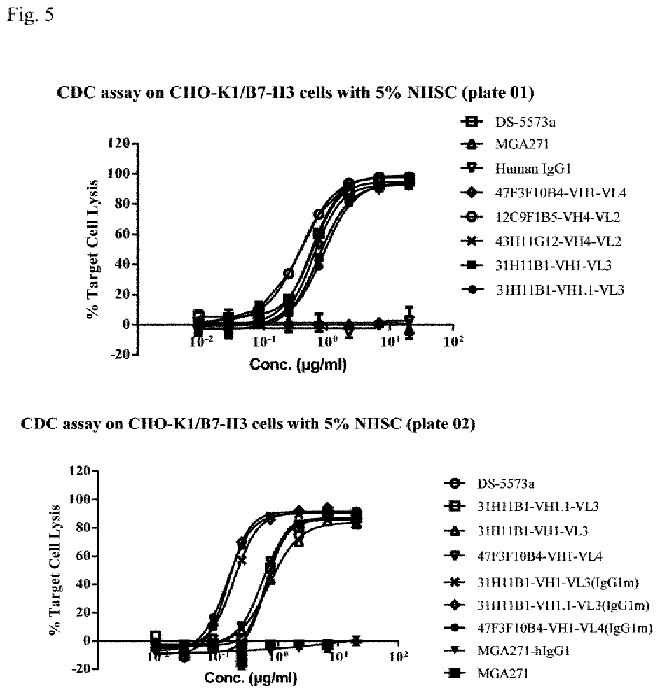

CHO-K1/B7-H3 Cell Binding by FACS To verify cell surface antigen binding of humanized antibodies, CHO-K1 cells expressing human B7-H3 were harvested and incubated with anti-B7-H3 mAbs, followed by goat anti-human IgG (H+L) secondary antibodies (Invitrogen). The samples were then analyzed with flow cytometry. The typical binding figures were shown in FIG. 4.

The FACS binding EC₅₀ values were summarized in Table 8. All humanized antibodies showed much higher binding affinity than the reference antibody of MGA271 (Patent NO: US 2012/0294796 A1, the heavy chain is IgG1m described above) and MGA271-hIgG1 (the heavy chain is wild type human IgG1 and other amino acid sequence is the same as MGA271).

TABLE 8
Humanization antibody FACS binding analysis

| Clone No. | FACS binding EC₅₀ value (nM) |
|---|---|
| 47F3F10B4-VH1-VL4 | 0.8884 |
| 47F3F10B4-VH2-VL4 | 0.7667 |
| 12C9F1B5-VH2.1-VL2 | 0.1528 |
| 31H11B1-VH1-VL3 | 0.7904 |
| 31H11B1-VH1.1-VL3 | 0.4053 |
| 12C9F1B5-VH4-VL2 | 0.3385 |
| 43H11G12-VH4-VL2 | 0.1554 |
| 31H11B1-VH1-VL3(IgG1m) | 0.4209 |
| 31H11B1-VH1.1-VL3(IgG1m) | 0.3342 |
| 47F3F10B4-VH1-VL4(IgG1m) | 0.2994 |
| MGA271 | 101.6 |
| MGA271-hIgG1 | 81.63 |
| human IgG | Not Applicable |

### Anti-B7-H3 Antibody Induced CDC Assay

CDC assay was conducted for the humanized antibodies. The target cell line, CHO-K1-overexpressing human B7-H3, was cultured and harvested, and it was seeded in 96-well plate at a specific cell density (5E3 cells/well). Antibody samples were added to the plate accordingly and the plate was incubated at 37° C./5% CO₂ for 30 min. Purified normal human serum complement (NHSC, SIGMA-ALDRICH) was then added to the plate and the plate was incubated further for 4 hours. The plate was taken out of the incubator and cells viability analyzed with Cell Titer-Glo® assay kit. The luminescence data was captured by PheraStar (BMG) for cell viability analysis. The CDC assay results in term of % target cell lysis versus candidate antibody concentration are shown in FIG. 5.

The EC₅₀ values of all the candidate humanized antibodies from CDC assay (two plates in one experiment) are summarized in table 9. The EC₅₀ value of the in-house synthesized positive control which has the identical amino acid sequence as DS-5573a (Patent NO: US 2013/0078234 A1) was used to calculate the relative activities for each candidate antibody on every assay plate (% Relative activity=(EC₅₀ of the positive control/EC₅₀ of the sample)*100%). Clone 31H11B1-V_{H}1-VL3(IgG1m), 31H11B1-V_{H}1.1-VL3(IgG1m), 47F3F10B4-V_{H}1-VL4(IgG1m) show more than 300% relative activity.

TABLE 9
Humanized antibody CDC induction analysis

| | | | % Relative |
|---|---|---|---|
| Plate No. | Sample ID | EC₅₀(μg/ml) | activity |
| plate 01 | 47F3F10B4-VH1-VL4 | 0.6187 | 96.10 |
| | 12C9F1B5-VH4-VL2 | 0.384 | 154.84 |
| | 43H11G12-VH4-VL2 | 0.5698 | 104.35 |
| | 31H11B1-VH1-VL3 | 0.7393 | 80.43 |
| | 31H11B1-VH1.1-VL3 | 0.874 | 68.03 |
| | DS-5573a | 0.5946 | 100.00 |
| | MGA271 | Not | Not |
| | | Applicable | Applicable |
| | Human IgG1 | Not | Not |
| | | Applicable | Applicable |
| plate 02 | 31H11B1-VH1.1-VL3 | 0.6361 | 98.70 |
| | 31H11B1-VH1-VL3(IgG1m) | 0.1827 | 343.62 |
| | 31H11B1-VH1.1-VL3(IgG1m) | 0.1457 | 430.89 |
| | 47F3F10B4-VH1-VL4(IgG1m) | 0.1412 | 444.62 |
| | 31H11B1-VH1-VL3 | 0.7064 | 88.87 |
| | 47F3F10B4-VH1-VL4 | 0.5494 | 114.27 |
| | DS-5573a | 0.6278 | 100.00 |
| | MGA271-hIgG1 | Not | Not |
| | | Applicable | Applicable |
| | MGA271 | Not | Not |
| | | Applicable | Applicable |

### Anti-B7-H3 Antibody Induced ADCC Assay

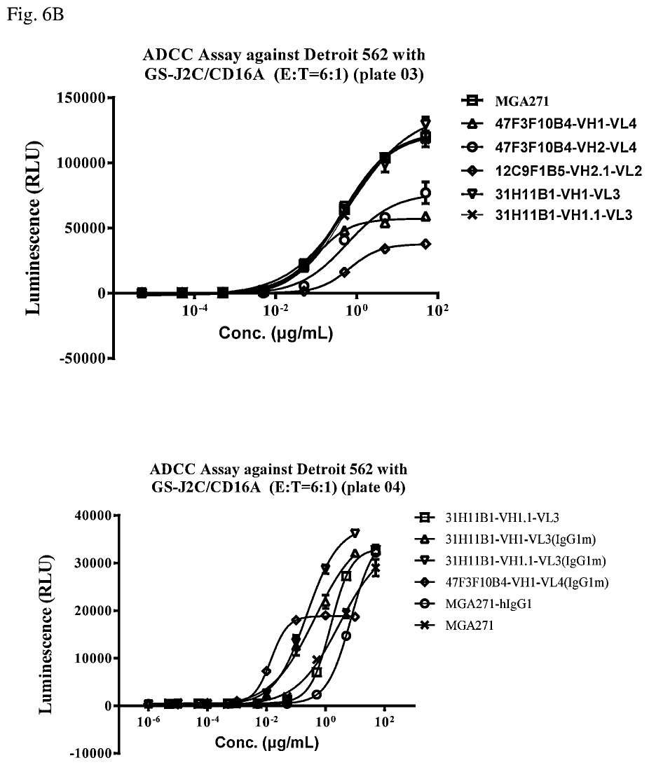

For the assay procedure, the target cell line, A549 (plate 01 and plate 02) and Detroit 562 (plate 03 and plate 04), were cultured, harvested and seeded into 96 well plates at a specific cell density (1E4 cells/well). The humanized antibody samples or positive control, which was in-house synthesized with the identical amino acid sequence of MGA-271 (Enoblituzumab) (Patent NO: US 2012/0294796 A1), were added to the plate and the plate was incubated at 37° C./5% CO₂ for 30 min ADCC reporter cell line (GS-J2C/CD16A), which was in-house structured referring to paper (see Paul et al, (2002) PLOS ONE 2014; 9(4): e95787), was used as the effector cells and added to the plates and incubated at the same condition for 6 hours. The assay plate was taken out and ADCC reporter cell line activation was analyzed with Bio-Glo® assay kit (Promega). The luminescence data was captured by PheraStar (BMG) for cell viability analysis. The absorbance data were captured by PheraStar (BMG) and analyzed by GraphPad Prism 6.0. The ADCC effect of humanized antibodies were compared. The ADCC assay results are shown in FIGS. 6A-6B.

The EC₅₀ values of the following humanized antibodies from ADCC assay are summarized in table 10. Clone 47F3F10B4-V_{H}1-VL4(IgG1m) shows the lowest EC₅₀ value in A549 cells (0.00959 μg/ml) and Detroit 562 cells (0.01419 μg/ml). The EC₅0 value of the in-house synthesized positive control which has the identical amino acid sequence as MGA-271 (Enoblituzumab) (Patent NO: US 2012/0294796 A1) was used to calculate the relative activities for each candidate antibody on every assay plate (% Relative activity=(EC₅₀ of the positive control/EC₅₀ of the sample)*100). 31H11B1-V_{H}1-VL3(IgG1m), 31H0B1-V_{H}1.1-VL3(gGlm) and 47F3F10B4-V_{H}1-VL4(IgG1m) show more than 1000% relative activity targeting A549 cells and more than 600% relative activity targeting Detroit 562 cells in ADCC assay

These results from in vitro assay provides a possible treatment for solid tumors (HNSCC, NSCLC), and the safety and efficacy of these antibodies need to be confirmed in further preclinical and clinical studies.

TABLE 10
Humanized antibody ADCC induction analysis

| | | | % Relative |
|---|---|---|---|
| Plate No. | Sample ID | EC₅₀(μg/ml) | activity |
| plate 01 | 47F3F10B4-VH1-VL4 | 0.0391 | 365.22 |
| | 47F3F10B4-VH2-VL4 | 0.1719 | 83.07 |
| | 12C9F1B5-VH2.1-VL2 | 0.6254 | 22.83 |
| | 31H11B1-VH1-VL3 | 0.2567 | 55.63 |
| | 31H11B1-VH1.1-VL3 | 0.2135 | 66.89 |
| | MGA271 | 0.1428 | 100.00 |
| plate 02 | 31H11B1-VH1.1-VL3 | 1.225 | 55.18 |
| | 31H11B1-VH1-VL3(IgG1m) | 0.06465 | 1045.63 |
| | 31H11B1-VH1.1-VL3(IgG1m) | 0.04581 | 1475.66 |
| | 47F3F10B4-VH1-VL4(IgG1m) | 0.00959 | 7049.01 |
| | MGA271-hIgG1 | 7.764 | 8.71 |
| | MGA271 | 0.676 | 100.00 |
| plate 03 | 47F3F10B4-VH1-VL4 | 0.09533 | 467.01 |
| | 47F3F10B4-VH2-VL4 | 0.5767 | 77.20 |
| | 12C9F1B5-VH2.1-VL2 | 0.6662 | 66.83 |
| | 31H11B1-VH1-VL3 | 0.6921 | 64.33 |
| | 31H11B1-VH1.1-VL3 | 0.5065 | 87.90 |
| | MGA271 | 0.4452 | 100.00 |
| plate 04 | 31H11B1-VH1.1-VL3 | 1.501 | 193.67 |
| | 31H11B1-VH1-VL3(IgG1m) | 0.4357 | 667.20 |
| | 31H11B1-VH1.1-VL3(IgG1m) | 0.2286 | 1271.65 |
| | 47F3F10B4-VH1-VL4(IgG1m) | 0.01419 | 20486.26 |
| | MGA271-hIgG1 | 7.297 | 39.84 |
| | MGA271 | 2.907 | 100.00 |

Listed below are some amino acid sequences and nucleic acid sequences mentioned herein. The underlined amino acid sequences are CDRH1, CDRH2, CDRH3, CDRL1, CDRL2, and CDRL3 respectively.

>47F3F10B4-VH (SEQ ID NO: 1)
EVQLQQSGPELVKPGASVKMSCKASGYTFTDHYMHWVKQSHGKSLEWIGYIFPNNGVS
GYNQNFKDKATLTVDKSSSTAYMDLRSLTPEDSAVYYCTRRQLGLGTMDYWGQGTSV
TVSS
(The underlined CDR amino acid sequences are
designated SEQ ID NOs: 2, 3, and 4, respectively)
>47F3F10B4-VL (SEQ ID NO: 5)
DIVLTQSPASLAVSLGQRATISCRASKSVSTSGYNYMHWYQQKPGQPPKLLIYLASNLES
GVPARFSGSGSGTDFTLNIHPVEEEDAATYYCQHSKELPWTFGGGTKLEIK
(The underlined CDR amino acid sequences are designated
SEQ ID NOs: 6, 7, and 8, respectively)
>12C9F1B5-VH (SEQ ID NO: 9)
QGQIQLVQSGPELKKPGETVKISCKASGFTNYGMNWVKQAPGKGLKWMGWINPYSGV
PTYADDFKGRFAFSLETSASTAYLQINNLKNEDTATYFCARDDGFHYTMDYWGQGTSV
TVSS
(The underlined CDR amino acid sequences are
designated SEQ ID NOs: 10, 11, and 12, respectively)
>12C9F1B5-VL (SEQ ID NO: 13)
DVVLTQTPLSLPVSLGDQASISCRSSQNLVHSNGITYLHWYLQKPGQSPKLLIYKVSNRF
SGVPDRFSGSGSGTDFTLTISRVEAEDLGVYFCSQSTLVPWTFGGGTKLEIK
(The underlined CDR amino acid sequences are designated
SEQ ID NOs: 14, 15, and 16, respectively)
>34A9C12-VH (SEQ ID NO: 17)
QGQIQLVQSGPELKKSGETVKISCKASGFTNYGINWVKQAPGKGLKWMGWINPYSGVP
TYADDFKGRFAFSLETSASTAYLQINNLKNEDSATYFCARDDGFHYTMDYWGQGTSVT
VSS
(The underlined CDR amino acid sequences are
designated SEQ ID NOs: 18, 19, and 20, respectively)
>34A9C12-VL (SEQ ID NO: 21)
DVVLIQTPLSLPVSLGDQASISCRSSQNLVHSNGITYLHWYLQKPGQSPKLLIYKVSNRFS
GVPDRFSGSGSGTDFTLKISRVEAEDLGVYFCSQSTLVPWTFGGGTKLEIK
(The underlined CDR amino acid sequences are designated
SEQ ID NOs: 22, 23, and 24, respectively)
>38D6A4-VH (SEQ ID NO: 25)
QIQLVQSGPELKKPGEAVKISCKASGSTFTDFGMNWVRQAPGKGLKWMGWINPYSGVP
TYADEFKGRFVFSLETSARVAYMQINDLKNEDTATYFCARDDGYHYTMDYWGQGTSV
TVSS
(The underlined CDR amino acid sequences are
designated SEQ ID NOs: 26, 27, and 28, respectively)
>38D6A4-VL (SEQ ID NO: 29)
DVVMTQTPLPLPVSLGDQASISCRSSQSLIHSNGITYLHWYLQKPGQSPKLLIYKVSNRFS
GVPDRFTGSGSGTDFTLKISRVEAEDLGVYFCSQSTHVPWTFGGGSKLEIR
(The underlined CDR amino acid sequences are designated
SEQ ID NOs: 30, 31, and 32, respectively)
>37D11F5B8-VH (SEQ ID NO: 33)
QIQLVQSGPELKKPGETVKISCKASGDTFRNNGMNWVKQAPGKGLKWMGWINPYSGV
PTYADDFKGRFAFSLETSASTAFLQINNLKNEDTATYFCARDDGYHYTMDFWGLGTSVT
VSS
(The underlined CDR amino acid sequences are designated
SEQ ID NOs: 34, 35, and 36, respectively)
>37D11F5B8-VL (SEQ ID NO: 37)
DVVMTQTPLSLPVSLGDQASISCRSSQSLVHSNGITYLHWYLQKPGQSPKLLTYKVSHRF
SGVPDRFSGSGSGTDFTLKISRVEAEDLGVYFCSQSTHVPWTFGGGTKLEIK
(The underlined CDR amino acid sequences are designated
SEQ ID NOs: 38, 39, and 40, respectively)
>37F11A10-VH (SEQ ID NO: 41)
QIQLVQSGPELKKPGETVKISCKASGDTFRNYGMNWVKQAPGKGLKWMGWINPYSGV
PTYADDFKGRFAFSLETSASTAFLQINNLKNEDTATYFCARDDGFHYTMDYWGLGTSVT
VSS
(The underlined CDR amino acid sequences are designated
SEQ ID NOs: 42, 43, and 44, respectively)
>37F11A10-VL (SEQ ID NO: 45)
DVVMTQTPLSLPVSLGDQASISCRSSQSLLHSNGITYLHWYLQKPGQSPKLLTYKVSHRF
SGVPDRFSGSGSGTDFTLKISRVEAEDLGVYFCSQSTHVPWTFGGGTKLEIR
(The underlined CDR amino acid sequences are designated
SEQ ID NOs: 46, 47, and 48, respectively)
>43H11G12-VH (SEQ ID NO: 49)
QIQLVQSGPELKKPGETVKISCKASGYNFRDFGMNWVKQAPGKGLKWMGWLNSYSGV
PTYADDFKGRFVLSLETSASTAYLQINNLINEDTATYFCGRDDGYNYTMNYWGQGTSV
TVSS
(The underlined CDR amino acid sequences are
designated SEQ ID NOs: 50, 51, and 52, respectively)
>43H11G12-VL (SEQ ID NO: 53)
DVVMTQAPLSLPVSLGDQASISCRSSQSLVHSNGITYLHWYLQRAGQSPQLLIYKVSNRF
SGVPDRFSGSGSGTDFTLKISRVEAEDLGVYFCSQSTHVPWTFGGGTKLEIK
(The underlined CDR amino acid sequences are designated
SEQ ID NOs: 54, 55, and 56, respectively)
>38F11B2-VH (SEQ ID NO: 57)
QIQLVQSGPELKKPGETVKISCKASGYTFIDYGMNWVKQAPGKGLKWMGWINIYSGVP
TYADDFKGRFAFSLETSANTAYLQINNLKNEDTATYFCARDDGYHYTMNYWGQGTSVT
VSS
(The underlined CDR amino acid sequences are
designated SEQ ID NOs: 58, 59, and 60, respectively)
>38F11B2-VL (SEQ ID NO: 61)
DIVMTQTPLSLPVSLGDQASISCRSSQSLVHSNGITYLHWYLQKSGQSPKLLIYKVSNRFS
GVPDRFSGSGSGTAFTLKISRVEAEDLGVYFCSQSTHVPWTFGGGTKLEIK
(The underlined CDR amino acid sequences are designated
SEQ ID NOs: 62, 63, and 64, respectively)
>47C7E3-VH (SEQ ID NO: 65)
QIQLVQSGPELKKPGETVKISCKASGYTFTNFGMNWVKQAPGKGLKWMGWIYTPSGVP
IYADDFKGRFAVSLETSASTAYLQINNLKNEDTATYFCARDDGYYYTMDYWGQGTSVT
VSS
(The underlined CDR amino acid sequences are
designated SEQ ID NOs: 66, 67 and 68, respectively)
>47C7E3-VL (SEQ ID NO: 69)
DDVMTQTPLSLPVSLGDHASISCRSSQSLVHSNGDTYLHWYLQKPGQSPKLLIYKVSNRF
SGVPDRFSGSGSGTDFTLKISRVEAEDLGVYFCSQSTHVPWTFGGGTKLEIK
(The underlined CDR amino acid sequences are designated
SEQ ID NOs: 70, 71, and 72, respectively)
>31H11B1-VH (SEQ ID NO: 73)
QVQLQQPGAELGKPGTSVKLSCKASGYTFTSYWMHWVKQRPGQGLEWIGHINPNSGST
NQNAKFKNKATLTVDESSSTAYMQLSTLTSEDSAVYYCTRSGSNYRRNYFDYWGQGTT
LTVSS
(The underlined CDR amino acid sequences are
designated SEQ ID NOs: 74, 75, and 76, respectively)
>31H11B1-VL (SEQ ID NO: 77)
DIQMTQSSSYLSVSLGGRVTITCKASDHINNWLAWYQQKPGNAPRLLISGATSLETGVPS
RFSGSGSGKDYTLSITSLQTEDVATYYCQQYWSTPLTFGAGTKLELK
(The underlined CDR amino acid sequences are designated
SEQ ID NOs: 78, 79, and 80, respectively)
The following protein sequences are humanized mAb sequences:
>47F3F10B4-VH1 (SEQ ID NO: 81)
QVQLVQSGAEVKKPGASVKVSCKASGYTFTDHYMHWVRQAPGQGLEWMGYIFPNNG
VSGYNQNFKDRVTMTRDTSISTAYMELSRLRSDDTAVYYCARRQLGLGTMDYWGQGT
LVTVSS
>47F3F10B4-VH2 (SEQ ID NO: 82)
QVQLVQSGAEVKKPGASVKVSCKASGYTFTDHYMHWVRQAPGQGLEWMGYIFPNNG
VSGYNQNFKDRVTMTRDTSTSTVYMELSSLRSEDTAVYYCARRQLGLGTMDYWGQGT
LVTVSS
>12C9F1B5-VH2.1 (SEQ ID NO: 83)
QVQLVQSGAEVKKPGASVKVSCKASGFTNYGMNWVRQAPGQGLEWMGWINPYSGVP
TYADDFKGRVTMTRDTSISTAYMELSRLRSDDTAVYYCARDDGFHYTMDYWGQGTLV
TVSS
>12C9F1B5-VH4 (SEQ ID NO: 84)
QVQLVQSGAEVKKPGASVKVSCKASGFTNYGMNWVRQAPGQRLEWMGWINPYSGVP
TYADDFKGRVTITRDTSASTAYMELSSLRSEDTAVYYCARDDGFHYTMDYWGQGTLVT
VSS
>43H11G12-VH4 (SEQ ID NO: 85)
QVQLVQSGAEVKKPGASVKVSCKASGYNFRDFGMNWVRQAPGQRLEWMGWLNSYSG
VPTYADDFKGRVTITRDTSASTAYMELSSLRSEDTAVYYCARDDGYNYTMNYWGQGT
LVTVSS
>31H11B1-VH1 (SEQ ID NO: 86)
QVQLVQSGAEVKKPGASVKVSCKASGYTFTSYWMHWVRQAPGQGLEWMGHINPNSG
STNQNAKFKNRVTMTRDTSISTAYMELSRLRSDDTAVYYCARSGSNYRRNYFDYWGQ
GTLVTVSS
>31H11B1-VH1.1 (SEQ ID NO: 87)
QVQLVQSGAEVKKPGASVKVSCKASGYTFTSYWMHWVRQAPGQGLEWMGHINPNSG
STNQNAKFKNRVTMTRDTSISTAYMELSRLRSDDTAVYYCTRSGSNYRRNYFDYWGQG
TLVTVSS
>47F3F10B4-VL4 (SEQ ID NO: 88)
DIQMTQSPSSLSASVGDRVTITCRASKSVSTSGYNYMHWYQQKPGKAPKLLIYLASNLE
SGVPSRFSGSGSGTDFTLTISSLQPEDFATYYCQHSKELPWTFGQGTKVEIK
>12C9F1B5-VL2 (SEQ ID NO: 89)
DIVMTQSPLSLPVTPGEPASISCRSSQNLVHSNGITYLHWYLQKPGQSPQLLIYKVSNRFS
GVPDRFSGSGSGTDFTLKISRVEAEDVGVYYCSQSTLVPWTFGQGTKVEIK
>43H11G12-VL2 (SEQ ID NO: 90)
DIVMTQSPLSLPVTPGEPASISCRSSQSLVHSNGITYLHWYLQKPGQSPQLLIYKVSNRFS
GVPDRFSGSGSGTDFTLKISRVEAEDVGVYYCSQSTHVPWTFGQGTKVEIK
>31H11B1-VL3 (SEQ ID NO: 91)
DIQMTQSPSSLSASVGDRVTITCKASDHINNWLAWYQQKPGKAPKLLIYGATSLETGVPS
RFSGSGSGTDFTFTISSLQPEDIATYYCQQYWSTPLTFGGGTKLEIK
Human kappa light chain constant region (SEQ ID NO: 92):
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Human IgG1 heavy chain constant region (SEQ ID NO: 93):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Human IgG1m sequence (SEQ ID NO: 94):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELVGG
PSVFLLPPKPKDTLMISRTPEVTCVVVDVSHEDPEVKFNWYVDGVEVHNAKTKPPEEQY
NSTLRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPLVLDSDGSFFLYSKLTVDK
SRWQQGNVFSCSVMHEALHNHYTQKSLSLSPGK

1. An isolated antibody or an antigen-binding fragment thereof, comprising:
I) a heavy chain variable domain (V_{H}) comprising:
1) a heavy chain determining region 1 (CDR1);
2) a heavy chain CDR2; and
3) a heavy chain CDR3; and
II) a light chain variable domain (VL) comprising:
1) a light chain CDR1;
2) a light chain CDR2; and
3) a light chain CDR3,
wherein:
1) the V_{H} comprises the heavy chain CDR1, CDR2, and CDR3 sequences having the amino acid sequences of SEQ ID NOs: 2, 3, and 4, respectively, and the VL comprises the light chain CDR1, CDR2, and CDR3 having the amino acid sequences of SEQ ID NOs: 6, 7, and 8, respectively;
2) the V_{H} comprises the heavy chain CDR1, CDR2, and CDR3 sequences having the amino acid sequences of SEQ ID NOs: 10, 11, and 12, respectively, and the VL comprises the light chain CDR1, CDR2, and CDR3 having the amino acid sequences of SEQ ID NOs: 14, 15, and 16, respectively;
3) the V_{H} comprises the heavy chain CDR1, CDR2, and CDR3 sequences having the amino acid sequences of SEQ ID NOs: 18, 19, and 20, respectively, and the VL comprises the light chain CDR1, CDR2, and CDR3 having the amino acid sequences of SEO ID NOs: 22, 23, and 24, respectively;
4) the V_{H} comprises the heavy chain CDR1, CDR2, and CDR3 sequences having the amino acid sequences of SEQ ID NOs: 26, 27, and 28, respectively, and the VL comprises the light chain CDR1, CDR2, and CDR3 having the amino acid sequences of SEQ ID NOs: 30, 31, and 32, respectively;
5) the V_{H} comprises the heavy chain CDR1, CDR2, and CDR3 sequences having the amino acid sequences of SEQ ID NOs: 34, 35, and 36, respectively, and the VL comprises the light chain CDR1, CDR2, and CDR3 having the amino acid sequences of SEQ ID NOs: 38, 39, and 40, respectively;
6) the V_{H} comprises the heavy chain CDR1, CDR2, and CDR3 sequences having the amino acid sequences of SEQ ID NOs: 42, 43, and 44, respectively, and the VL comprises the light chain CDR1, CDR2, and CDR3 having the amino acid sequences of SEQ ID NOs:46, 47, and 48, respectively;
7) the V_{H} comprises the heavy chain CDR1, CDR2, and CDR3 sequences having the amino acid sequences of SEQ ID NOs: 50, 51, and 52, respectively, and the VL comprises the light chain CDR1, CDR2, and CDR3 having the amino acid sequences of SEQ ID NOs: 54, 55, and 56, respectively;
8) the V_{H} comprises the heavy chain CDR1, CDR2, and CDR3 sequences having the amino acid sequences of SEQ ID NOs: 58, 59, and 60, respectively, and the VL comprises the light chain CDR1, CDR2, and CDR3 having the amino acid sequences of SEQ ID NOs: 62, 63, and 64, respectively;
9) the V_{H} comprises the heavy chain CDR1, CDR2, and CDR3 sequences having the amino acid sequences of SEQ ID NOs: 66, 67, and 68, respectively, and the VL comprises the light chain CDR1, CDR2, and CDR3 having the amino acid sequences of SEQ ID NOs:70, 71, and 72, respectively; or
10) the V_{H} comprises the heavy chain CDR1, CDR2, and CDR3 sequences having the amino acid sequences of SEQ ID NOs: 74, 75, and 76, respectively, and the VL comprises the light chain CDR1, CDR2, and CDR3 having the amino acid sequences of SEQ ID NOs:78, 79, and 80, respectively,
wherein the antibody or antigen-binding fragment thereof is capable of specifically binding to a B7-H3.

2. (canceled)

3. The isolated antibody or antigen-binding fragment thereof of claim 1, wherein the V_{H} comprises an amino acid sequence that is at least 90% identical to a sequence selected from the group consisting of SEQ ID NOs:1, 9, 17, 25, 33, 41, 49, 57, 65, and 73, and the VL comprises an amino acid sequence that is at least 90% identical to a sequence selected from the group consisting of SEQ ID NOs: 5, 13, 21, 29, 37, 45, 53, 61, 69, and 77, respectively.

4. (canceled)

5. The isolated antibody or antigen-binding fragment thereof of claim 1, wherein:
1) the V_{H} comprises an amino acid sequence of SEQ ID NO: 1, and the VL comprises an amino acid sequence of SEQ ID NO: 5;
2) the V_{H} comprises an amino acid sequence of SEQ ID NO: 9, and the VL comprises an amino acid sequence of SEQ ID NO: 13;
3) the V_{H} comprises an amino acid sequence of SEQ ID NO: 17, and the VL comprises an amino acid sequence of SEQ ID NO: 21;
4) the V_{H} comprises an amino acid sequence of SEQ ID NO: 25, and the VL comprises an amino acid sequence of SEQ ID NO: 29;
5) the V_{H} comprises an amino acid sequence of SEQ ID NO: 33, and the VL comprises an amino acid sequence of SEQ ID NO: 37;
6) the V_{H} comprises an amino acid sequence of SEQ ID NO: 41, and the VL comprises an amino acid sequence of SEQ ID NO: 45;
7) the V_{H} comprises an amino acid sequence of SEQ ID NO: 49, and the VL comprises an amino acid sequence of SEQ ID NO: 53;
8) the V_{H} comprises an amino acid sequence of SEQ ID NO: 57, and the VL comprises an amino acid sequence of SEQ ID NO:61;
9) the V_{H} comprises an amino acid sequence of SEQ ID NO: 65, and the VL comprises an amino acid sequence of SEQ ID NO: 69; or
10) the V_{H} comprises an amino acid sequence of SEQ ID NO: 73, and the VL comprises an amino acid sequence of SEQ ID NO: 77.

6-7. (canceled)

8. The isolated antibody or antigen-binding fragment thereof of claim 1, wherein the antibody is a mouse, chimeric, humanized or human antibody.

9. The isolated antibody or antigen-binding fragment thereof of claim 8, wherein the humanized antibody comprises a V_{H} comprising an amino acid sequence selected from the group consisting of SEQ ID NOs: 81-87, and a VL comprising an amino acid sequence selected from the group consisting of SEQ ID NOs: 88-91.

10. The isolated antibody or antigen-binding fragment thereof of claim 9, wherein the humanized antibody comprises:
1) a V_{H} comprising an amino acid sequence of SEQ ID NO: 81, and a VL comprising an amino acid sequence of SEQ ID NO: 88;
2) a V_{H} comprising an amino acid sequence of SEQ ID NO: 82, and a VL comprising an amino acid sequence of SEQ ID NO: 88;
3) a V_{H} comprising an amino acid sequence of SEQ ID NO: 83, and a VL comprising an amino acid sequence of SEQ ID NO: 89;
4) a V_{H} comprising an amino acid sequence of SEQ ID NO: 84, and a VL comprising an amino acid sequence of SEQ ID NO: 89;
5) a V_{H} comprising an amino acid sequence of SEQ ID NO: 85, and a VL comprising an amino acid sequence of SEQ ID NO: 90;
6) a V_{H} comprising an amino acid sequence of SEQ ID NO: 86, and a VL comprising an amino acid sequence of SEQ ID NO: 91; or
7) a V_{H} comprising an amino acid sequence of SEQ ID NO: 8687, and a VL comprising an amino acid sequence of SEQ ID NO: 91.

11. The isolated antibody or antigen-binding fragment thereof of claim 1, wherein the V_{H} is fused to a heavy chain constant region of an immunoglobulin.

12. The isolated antibody or antigen-binding fragment thereof of claim 11, wherein the heavy chain constant region is from human IgG1.

13. The isolated antibody or antigen-binding fragment thereof of any claim 12, wherein the heavy chain constant region comprises a modification that enhances the ADCC effect of the antibody, wherein the heavy chain constant region comprises mutations K214R, L235V, F243L, R292P, Y300L, D356E, L358M and P396L.

14. (canceled)

15. The isolated antibody or antigen-binding fragment thereof of claim 13 wherein the heavy chain constant region comprises an amino acid sequence of SEQ ID NO: 93 or 94.

16. The isolated antibody or antigen-binding fragment thereof of claim 1, wherein the VL is fused to a light chain constant region (CL) of an immunoglobulin, wherein the light chain constant region comprises an amino acid sequence of SEQ ID NO: 92.

17. (canceled)

18. The isolated antibody or antigen-binding fragment thereof of claim 1, conjugated to a toxin or a chemotherapeutic agent.

19. The isolated antibody or antigen binding fragment of claim 18, wherein the toxin is a Pseudomonas exotoxin (PE), ricin, abrin, diphtheria toxin, ribotoxin, saporin, calicheamicin, or a botulinum toxin; or,
the chemotherapeutic agent is Monomethyl Auristatin E or a maytansinoid.

20. (canceled)

21. A bispecific antibody comprising the isolated antibody or an antigen-binding fragment of claim 1 and a second antibody moiety, wherein the second antibody moiety is able to specifically bind to an antigen other than B7-H3.

22-23. (canceled)

24. The bispecific antibody of claim 21, wherein the second antibody moiety is a Fab, a Fab′, a (Fab′)₂, an Fv, a single chain Fv (scFv), an scFv-scFv, a minibody, a diabody, an sdAb, or an antibody mimetic.

25. A pharmaceutical composition f comprising:
the isolated antibody or antigen-binding fragment thereof of claim 1 and
a pharmaceutically acceptable carrier.

26-27. (canceled)

28. A method of treating cancer comprising administrating to a subject in need thereof a therapeutically effective amount of the isolated antibody or antigen-binding fragment thereof of claim 1.

29. The method of claim 28, wherein the cancer is a B7-H3-expressing cancer.

30-35. (canceled)

36. A vector encoding the antibody or antigen binding fragment of claim 1.

37. A host cell comprising the vector of claim 36.


SEQUENCE LISTING (94 sequences)

SEQ ID NO: 1 - protein, 119 aa, synthetic construct
EVQLQQSGPELVKPGASVKMSCKASGYTFTDHYMHWVKQSHGKSLEWIGYIFPNNGVSGYNQNFKDKATLTVDKSSSTAYMDLRSLTPEDSAVYYCTRRQLGLGTMDYWGQGTSVTVSS

SEQ ID NO: 2 - protein, 5 aa, synthetic construct
DHYMH

SEQ ID NO: 3 - protein, 17 aa, synthetic construct
YIFPNNGVSGYNQNFKD

SEQ ID NO: 4 - protein, 10 aa, synthetic construct
RQLGLGTMDY

SEQ ID NO: 5 - protein, 111 aa, synthetic construct
DIVLTQSPASLAVSLGQRATISCRASKSVSTSGYNYMHWYQQKPGQPPKLLIYLASNLESGVPARFSGSGSGTDFTLNIHPVEEEDAATYYCQHSKELPWTFGGGTKLEIK

SEQ ID NO: 6 - protein, 15 aa, synthetic construct
RASKSVSTSGYNYMH

SEQ ID NO: 7 - protein, 7 aa, synthetic construct
LASNLES

SEQ ID NO: 8 - protein, 9 aa, synthetic construct
QHSKELPWT

SEQ ID NO: 9 - protein, 119 aa, synthetic construct
QGQIQLVQSGPELKKPGETVKISCKASGFTNYGMNWVKQAPGKGLKWMGWINPYSGVPTYADDFKGRFAFSLETSASTAYLQINNLKNEDTATYFCARDDGFHYTMDYWGQGTSVTVSS

SEQ ID NO: 10 - skipped

SEQ ID NO: 11 - protein, 17 aa, synthetic construct
WINPYSGVPTYADDFKG

SEQ ID NO: 12 - protein, 10 aa, synthetic construct
DDGFHYTMDY

SEQ ID NO: 13 - protein, 112 aa, synthetic construct
DVVLTQTPLSLPVSLGDQASISCRSSQNLVHSNGITYLHWYLQKPGQSPKLLIYKVSNRFSGVPDRFSGSGSGTDFTLTISRVEAEDLGVYFCSQSTLVPWTFGGGTKLEIK

SEQ ID NO: 14 - protein, 16 aa, synthetic construct
RSSQNLVHSNGITYLH

SEQ ID NO: 15 - protein, 7 aa, synthetic construct
KVSNRFS

SEQ ID NO: 16 - protein, 9 aa, synthetic construct
SQSTLVPWT

SEQ ID NO: 17 - protein, 119 aa, synthetic construct
QGQIQLVQSGPELKKSGETVKISCKASGFTNYGINWVKQAPGKGLKWMGWINPYSGVPTYADDFKGRFAFSLETSASTAYLQINNLKNEDSATYFCARDDGFHYTMDYWGQGTSVTVSS

SEQ ID NO: 18 - skipped

SEQ ID NO: 19 - protein, 17 aa, synthetic construct
WINPYSGVPTYADDFKG

SEQ ID NO: 20 - protein, 10 aa, synthetic construct
DDGFHYTMDY

SEQ ID NO: 21 - protein, 112 aa, synthetic construct
DVVLIQTPLSLPVSLGDQASISCRSSQNLVHSNGITYLHWYLQKPGQSPKLLIYKVSNRFSGVPDRFSGSGSGTDFTLKISRVEAEDLGVYFCSQSTLVPWTFGGGTKLEIK

SEQ ID NO: 22 - protein, 16 aa, synthetic construct
RSSQNLVHSNGITYLH

SEQ ID NO: 23 - protein, 7 aa, synthetic construct
KVSNRFS

SEQ ID NO: 24 - protein, 9 aa, synthetic construct
SQSTLVPWT

SEQ ID NO: 25 - protein, 119 aa, synthetic construct
QIQLVQSGPELKKPGEAVKISCKASGSTFTDFGMNWVRQAPGKGLKWMGWINPYSGVPTYADEFKGRFVFSLETSARVAYMQINDLKNEDTATYFCARDDGYHYTMDYWGQGTSVTVSS

SEQ ID NO: 26 - protein, 5 aa, synthetic construct
DFGMN

SEQ ID NO: 27 - protein, 17 aa, synthetic construct
WINPYSGVPTYADEFKG

SEQ ID NO: 28 - protein, 10 aa, synthetic construct
DDGYHYTMDY

SEQ ID NO: 29 - protein, 112 aa, synthetic construct
DVVMTQTPLPLPVSLGDQASISCRSSQSLIHSNGITYLHWYLQKPGQSPKLLIYKVSNRFSGVPDRFTGSGSGTDFTLKISRVEAEDLGVYFCSQSTHVPWTFGGGSKLEIR

SEQ ID NO: 30 - protein, 16 aa, synthetic construct
RSSQSLIHSNGITYLH

SEQ ID NO: 31 - protein, 7 aa, synthetic construct
KVSNRFS

SEQ ID NO: 32 - protein, 9 aa, synthetic construct
SQSTHVPWT

SEQ ID NO: 33 - protein, 119 aa, synthetic construct
QIQLVQSGPELKKPGETVKISCKASGDTFRNNGMNWVKQAPGKGLKWMGWINPYSGVPTYADDFKGRFAFSLETSASTAFLQINNLKNEDTATYFCARDDGYHYTMDFWGLGTSVTVSS

SEQ ID NO: 34 - protein, 5 aa, synthetic construct
NNGMN

SEQ ID NO: 35 - protein, 17 aa, synthetic construct
WINPYSGVPTYADDFKG

SEQ ID NO: 36 - protein, 10 aa, synthetic construct
DDGYHYTMDF

SEQ ID NO: 37 - protein, 112 aa, synthetic construct
DVVMTQTPLSLPVSLGDQASISCRSSQSLVHSNGITYLHWYLQKPGQSPKLLTYKVSHRFSGVPDRFSGSGSGTDFTLKISRVEAEDLGVYFCSQSTHVPWTFGGGTKLEIK

SEQ ID NO: 38 - protein, 16 aa, synthetic construct
RSSQSLVHSNGITYLH

SEQ ID NO: 39 - protein, 7 aa, synthetic construct
KVSHRFS

SEQ ID NO: 40 - protein, 9 aa, synthetic construct
SQSTHVPWT

SEQ ID NO: 41 - protein, 119 aa, synthetic construct
QIQLVQSGPELKKPGETVKISCKASGDTFRNYGMNWVKQAPGKGLKWMGWINPYSGVPTYADDFKGRFAFSLETSASTAFLQINNLKNEDTATYFCARDDGFHYTMDYWGLGTSVTVSS

SEQ ID NO: 42 - protein, 5 aa, synthetic construct
NYGMN

SEQ ID NO: 43 - protein, 17 aa, synthetic construct
WINPYSGVPTYADDFKG

SEQ ID NO: 44 - protein, 10 aa, synthetic construct
DDGFHYTMDY

SEQ ID NO: 45 - protein, 112 aa, synthetic construct
DVVMTQTPLSLPVSLGDQASISCRSSQSLLHSNGITYLHWYLQKPGQSPKLLTYKVSHRFSGVPDRFSGSGSGTDFTLKISRVEAEDLGVYFCSQSTHVPWTFGGGTKLEIR

SEQ ID NO: 46 - protein, 16 aa, synthetic construct
RSSQSLLHSNGITYLH

SEQ ID NO: 47 - protein, 7 aa, synthetic construct
KVSHRFS

SEQ ID NO: 48 - protein, 9 aa, synthetic construct
SQSTHVPWT

SEQ ID NO: 49 - protein, 119 aa, synthetic construct
QIQLVQSGPELKKPGETVKISCKASGYNFRDFGMNWVKQAPGKGLKWMGWLNSYSGVPTYADDFKGRFVLSLETSASTAYLQINNLINEDTATYFCGRDDGYNYTMNYWGQGTSVTVSS

SEQ ID NO: 50 - protein, 5 aa, synthetic construct
DFGMN

SEQ ID NO: 51 - protein, 5 aa, synthetic construct
DFGMN

SEQ ID NO: 52 - protein, 10 aa, synthetic construct
DDGYNYTMNY

SEQ ID NO: 53 - protein, 112 aa, synthetic construct
DVVMTQAPLSLPVSLGDQASISCRSSQSLVHSNGITYLHWYLQRAGQSPQLLIYKVSNRFSGVPDRFSGSGSGTDFTLKISRVEAEDLGVYFCSQSTHVPWTFGGGTKLEIK

SEQ ID NO: 54 - protein, 16 aa, synthetic construct
RSSQSLVHSNGITYLH

SEQ ID NO: 55 - protein, 7 aa, synthetic construct
KVSNRFS

SEQ ID NO: 56 - protein, 9 aa, synthetic construct
SQSTHVPWT

SEQ ID NO: 57 - protein, 119 aa, synthetic construct
QIQLVQSGPELKKPGETVKISCKASGYTFIDYGMNWVKQAPGKGLKWMGWINIYSGVPTYADDFKGRFAFSLETSANTAYLQINNLKNEDTATYFCARDDGYHYTMNYWGQGTSVTVSS

SEQ ID NO: 58 - protein, 5 aa, synthetic construct
DYGMN

SEQ ID NO: 59 - protein, 17 aa, synthetic construct
WINIYSGVPTYADDFKG

SEQ ID NO: 60 - protein, 10 aa, synthetic construct
DDGYHYTMNY

SEQ ID NO: 61 - protein, 112 aa, synthetic construct
DIVMTQTPLSLPVSLGDQASISCRSSQSLVHSNGITYLHWYLQKSGQSPKLLIYKVSNRFSGVPDRFSGSGSGTAFTLKISRVEAEDLGVYFCSQSTHVPWTFGGGTKLEIK

SEQ ID NO: 62 - protein, 16 aa, synthetic construct
RSSQSLVHSNGITYLH

SEQ ID NO: 63 - protein, 7 aa, synthetic construct
KVSNRFS

SEQ ID NO: 64 - protein, 9 aa, synthetic construct
SQSTHVPWT

SEQ ID NO: 65 - protein, 119 aa, synthetic construct
QIQLVQSGPELKKPGETVKISCKASGYTFTNFGMNWVKQAPGKGLKWMGWIYTPSGVPIYADDFKGRFAVSLETSASTAYLQINNLKNEDTATYFCARDDGYYYTMDYWGQGTSVTVSS

SEQ ID NO: 66 - protein, 5 aa, synthetic construct
NFGMN

SEQ ID NO: 67 - protein, 17 aa, synthetic construct
WIYTPSGVPIYADDFKG

SEQ ID NO: 68 - protein, 10 aa, synthetic construct
DDGYYYTMDY

SEQ ID NO: 69 - protein, 112 aa, synthetic construct
DDVMTQTPLSLPVSLGDHASISCRSSQSLVHSNGDTYLHWYLQKPGQSPKLLIYKVSNRFSGVPDRFSGSGSGTDFTLKISRVEAEDLGVYFCSQSTHVPWTFGGGTKLEIK

SEQ ID NO: 70 - protein, 16 aa, synthetic construct
RSSQSLVHSNGDTYLH

SEQ ID NO: 71 - protein, 7 aa, synthetic construct
KVSNRFS

SEQ ID NO: 72 - protein, 9 aa, synthetic construct
SQSTHVPWT

SEQ ID NO: 73 - protein, 121 aa, synthetic construct
QVQLQQPGAELGKPGTSVKLSCKASGYTFTSYWMHWVKQRPGQGLEWIGHINPNSGSTNQNAKFKNKATLTVDESSSTAYMQLSTLTSEDSAVYYCTRSGSNYRRNYFDYWGQGTTLTVSS

SEQ ID NO: 74 - protein, 5 aa, synthetic construct
SYWMH

SEQ ID NO: 75 - protein, 17 aa, synthetic construct
HINPNSGSTNQNAKFKN

SEQ ID NO: 76 - protein, 12 aa, synthetic construct
SGSNYRRNYFDY

SEQ ID NO: 77 - protein, 107 aa, synthetic construct
DIQMTQSSSYLSVSLGGRVTITCKASDHINNWLAWYQQKPGNAPRLLISGATSLETGVPSRFSGSGSGKDYTLSITSLQTEDVATYYCQQYWSTPLTFGAGTKLELK

SEQ ID NO: 78 - protein, 11 aa, synthetic construct
KASDHINNWLA

SEQ ID NO: 79 - protein, 7 aa, synthetic construct
GATSLET

SEQ ID NO: 80 - protein, 9 aa, synthetic construct
QQYWSTPLT

SEQ ID NO: 81 - protein, 119 aa, synthetic construct
QVQLVQSGAEVKKPGASVKVSCKASGYTFTDHYMHWVRQAPGQGLEWMGYIFPNNGVSGYNQNFKDRVTMTRDTSISTAYMELSRLRSDDTAVYYCARRQLGLGTMDYWGQGTLVTVSS

SEQ ID NO: 82 - protein, 119 aa, synthetic construct
QVQLVQSGAEVKKPGASVKVSCKASGYTFTDHYMHWVRQAPGQGLEWMGYIFPNNGVSGYNQNFKDRVTMTRDTSTSTVYMELSSLRSEDTAVYYCARRQLGLGTMDYWGQGTLVTVSS

SEQ ID NO: 83 - protein, 117 aa, synthetic construct
QVQLVQSGAEVKKPGASVKVSCKASGFTNYGMNWVRQAPGQGLEWMGWINPYSGVPTYADDFKGRVTMTRDTSISTAYMELSRLRSDDTAVYYCARDDGFHYTMDYWGQGTLVTVSS

SEQ ID NO: 84 - protein, 117 aa, synthetic construct
QVQLVQSGAEVKKPGASVKVSCKASGFTNYGMNWVRQAPGQRLEWMGWINPYSGVPTYADDFKGRVTITRDTSASTAYMELSSLRSEDTAVYYCARDDGFHYTMDYWGQGTLVTVSS

SEQ ID NO: 85 - protein, 119 aa, synthetic construct
QVQLVQSGAEVKKPGASVKVSCKASGYNFRDFGMNWVRQAPGQRLEWMGWLNSYSGVPTYADDFKGRVTITRDTSASTAYMELSSLRSEDTAVYYCARDDGYNYTMNYWGQGTLVTVSS

SEQ ID NO: 86 - protein, 121 aa, synthetic construct
QVQLVQSGAEVKKPGASVKVSCKASGYTFTSYWMHWVRQAPGQGLEWMGHINPNSGSTNQNAKFKNRVTMTRDTSISTAYMELSRLRSDDTAVYYCARSGSNYRRNYFDYWGQGTLVTVSS

SEQ ID NO: 87 - protein, 121 aa, synthetic construct
QVQLVQSGAEVKKPGASVKVSCKASGYTFTSYWMHWVRQAPGQGLEWMGHINPNSGSTNQNAKFKNRVTMTRDTSISTAYMELSRLRSDDTAVYYCTRSGSNYRRNYFDYWGQGTLVTVSS

SEQ ID NO: 88 - protein, 111 aa, synthetic construct
DIQMTQSPSSLSASVGDRVTITCRASKSVSTSGYNYMHWYQQKPGKAPKLLIYLASNLESGVPSRFSGSGSGTDFTLTISSLQPEDFATYYCQHSKELPWTFGQGTKVEIK

SEQ ID NO: 89 - protein, 112 aa, synthetic construct
DIVMTQSPLSLPVTPGEPASISCRSSQNLVHSNGITYLHWYLQKPGQSPQLLIYKVSNRFSGVPDRFSGSGSGTDFTLKISRVEAEDVGVYYCSQSTLVPWTFGQGTKVEIK

SEQ ID NO: 90 - protein, 112 aa, synthetic construct
DIVMTQSPLSLPVTPGEPASISCRSSQSLVHSNGITYLHWYLQKPGQSPQLLIYKVSNRFSGVPDRFSGSGSGTDFTLKISRVEAEDVGVYYCSQSTHVPWTFGQGTKVEIK

SEQ ID NO: 91 - protein, 107 aa, synthetic construct
DIQMTQSPSSLSASVGDRVTITCKASDHINNWLAWYQQKPGKAPKLLIYGATSLETGVPSRFSGSGSGTDFTFTISSLQPEDIATYYCQQYWSTPLTFGGGTKLEIK

SEQ ID NO: 92 - protein, 107 aa, synthetic construct [sequence omitted]

SEQ ID NO: 93 - protein, 330 aa, synthetic construct [sequence omitted]

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK